(19) United States
(12) Patent Application Publication
(10) Pub. No.: US 2026/0021052 A1
(43) Pub. Date: Jan. 22, 2026

(54) PREPARATION OF POLYPEPTIDE FORMULATIONS USING VAPOR PHASE DEPOSITION

(71) Applicant: Applied Materials, Inc., Santa Clara, CA (US)

(72) Inventors: Fei Wang, Fremont, CA (US); Miaojun Wang, Santa Clara, CA (US); Pravin K. Narwankar, Sunnyvale, CA (US)

(21) Appl. No.: 19/274,209

(22) Filed: Jul. 18, 2025

Related U.S. Application Data
(60) Provisional application No. 63/673,617, filed on Jul. 19, 2024.

(51) Int. Cl. A61K 9/50; A61K 9/00; A61K 39/395; C07K 16/00

(52) U.S. Cl. CPC A61K 9/501; A61K 9/0024; A61K 9/5089; A61K 39/39591; C07K 16/00

(57) ABSTRACT

The disclosure is directed at improved polypeptide formulations comprising a coated particle comprising a polypeptide-containing core and a coating layer enclosing the polypeptide-containing core. Methods for manufacturing such a polypeptide formulations are also provided.

### CROSS-REFERENCE TO RELATED APPLICATIONS

This application claims priority to U.S. Application Ser. No. 63/673,617, filed on Jul. 19, 2024. The disclosure of the prior application is considered part of the disclosure of this application, and is incorporated in its entirety into this application.

### TECHNICAL FIELD

The present disclosure is in the field of polypeptide formulations.

### BACKGROUND

The desire to improve patient convenience and compliance with polypeptide drug products (e.g., therapeutic polypeptides, antibodies, and antibody-drug-conjugates) has led to a movement away from intravenous (i.v.) administration to subcutaneous (s.c.) injection. Because conventional s.c. injections generally have an injection volume of 2 ml or less and because many polypeptide drugs require a relatively high dose (e.g., 100 mg/dose) there is a need to develop high concentration formulations. Pharmaceutical development challenges for such formulations include high solution viscosity, polypeptide aggregation and polypeptide instability.

### SUMMARY

In one aspect, the disclosure provides high concentration polypeptide formulations comprising coated particles comprising a polypeptide-containing core and an inorganic oxide coating layer enclosing the particle. The inorganic oxide layer can be applied by vapor phase deposition and can be composed of several individual layers, for example, a silicon oxide layer and a mixed oxide layer. The coated particles may have a reduced surface charge as compared to the uncoated particles. The coated particles may have increased hydrophilicity as compared to the uncoated particles. The resulting coated particles can be dispersed in the water to form a high polypeptide content suspension. The high polypeptide content suspension may have a reduced viscosity as compared to formulations prepared with uncoated particles or suspensions containing the same amount of polypeptide in the same volume. The methods described herein can be used to create high concentration antibody formulations (HCAF) and long acting injectable (LAI) formulations.

In one aspect, the disclosure provides a long acting injectable formulations comprising coated particles comprising a polypeptide-containing core and an inorganic oxide coating layer that provides a controlled release profile. The coating inorganic oxide layer can be applied by vapor phase deposition and can be composed of several individual layers, for example, a silicon oxide layer and a mixed oxide layer. The coated particles may have a slower polypeptide release rate as compared with uncoated particles.

The process described herein for preparing LAI polypeptide formulations is simpler than many conventional methods for preparing LAI polypeptide formulations. FIGS. 5A-5B show a comparison of an example of a traditional LAI manufacturing process based on preparation of polypeptide microspheres (FIG. 5A) and an example of the methods described herein (FIG. 5B). The microsphere-based method includes at least six steps and requires large volumes of solvents (e.g., water and oil). By comparison, the methods described herein simplify the process and reduce solid, liquid, and energy waste.

In addition, LAI formulations containing coated particles described herein can have reduced burst release and can provide more stable plasma levels upon administration to a patient. Further, the coating layer can stabilize polypeptide, reducing the cold chain requirement common for traditional polypeptide formulations.

To create a high concentration (i.e., high polypeptide content) polypeptide formulation, the coated polypeptide particles are dispersed in an aqueous composition (e.g., physiological saline) as a suspension ready for subcutaneous injection. In the high concentration polypeptide formulation, the polypeptide (e.g., antibody) can be present at a concentration from about 50-500 mg/ml, 50-400 mg/ml, 100-500 mg/ml or 100-400 mg/ml. The aqueous formulations can include additional excipients and other components suitable for subcutaneous injection formulations or depot formulations.

The coating layer completely encloses the polypeptide-containing particle and can be composed of one or more inorganic oxide layers and/or one or more mixed inorganic oxide layers. The coating layer can also include one or more polymer layers.

In one aspect, the disclosure is related to a polypeptide formulation, comprising:
(a) a coated particle comprising a core comprising a polypeptide, and a coating layer comprising an inorganic oxide layer enclosing the core; and
(b) water,
   wherein the inorganic oxide layer comprises an inorganic oxide that comprises aluminum, zinc, silicon, and/or titanium, wherein the polypeptide formulation comprises more than 100 mg/ml polypeptide.

In some embodiments, the polypeptide formulation comprises more than 150 mg/ml polypeptide.

In some embodiments, the polypeptide formulation comprises more than 100 mg/ml coated particles.

In some embodiments, the coating layer is 0.1 nm-120 nm thick.

In some embodiments, the coating layer is 5 nm-15 nm thick.

In some embodiments, the coating layer is conformal and pin-hole free.

In some embodiments, the coated particle comprises 1-20% wt/wt inorganic oxide.

In some embodiments, the core consists of a polypeptide.

In some embodiments, the coated particle consists of the core and the coating layer.

In some embodiments, the coated particles have a D50 on a volume average basis of 100 nm-30 micrometers.

In some embodiments, the coated particles have a D50 on a volume average basis of 1-25 μm.

In some embodiments, the coated particles have a median particle size, on a volume average basis, between 0.1 μm and 20 μm.

In some embodiments, the polypeptide is selected from the group consisting of a growth factor, an antigen, an antibody, and an antibody drug conjugate (ADC).

In some embodiments, the polypeptide is an antibody.

In some embodiments, the coating increases the stability of the polypeptide.

In some embodiments, the coating reduces the viscosity of the formulation, as compared to a formulation containing uncoated particles.

In some embodiments, the coating slows the release rate of the polypeptide, as compared to a formulation containing uncoated particles.

In some embodiments, the coated particle has increased hydrophilicity compared to an uncoated core.

In some embodiments, the coated particle has increased flowability compared to an uncoated core.

In one aspect, the disclosure is related to a method of preparing coated particles comprising a core comprising a polypeptide and an inorganic oxide coating, the method comprising the sequential steps of:
(a) loading particles comprising a polypeptide and an excipient into a chamber of a reactor;
(b) performing a first number of first cycles, wherein each first cycle comprises steps (b1)-(b4):
(b1) applying a vaporous or gaseous first inorganic oxide precursor to the particles in the reactor by pulsing the vaporous or gaseous first inorganic oxide precursor into the reactor;
(b2) purging using an inert gas or performing one or more pump-purge cycles of the reactor using an inert gas;
(b3) applying a vaporous or gaseous oxidant to the particles in the reactor by pulsing the oxidant into the reactor;
(b4) purging using an inert gas or performing one or more pump-purge cycles of the reactor using an inert gas;
(c) performing a second number of second cycles, wherein each second cycle comprises steps (c1)-(c4):
(c1) applying a vaporous or gaseous second inorganic oxide precursor to the particles in the reactor by pulsing the vaporous or gaseous second inorganic oxide precursor into the reactor;
(c2) purging using an inert gas or performing one or more pump-purge cycles of the reactor using an inert gas;
(c3) applying a vaporous or gaseous oxidant to the particles in the reactor by pulsing the oxidant into the reactor;
(c4) purging using an inert gas or performing one or more pump-purge cycles of the reactor using an inert gas; and
(d) repeating steps (b)-(c) at least once;
wherein, the first inorganic oxide precursor and second inorganic oxide precursor are different; in each repeat of step (b) the first number is independently selected from 1-10 or 1-20; and in each repeat of step (c) the second number is independently selected from 1-10 or 1-20.

In some embodiments, the first inorganic oxide precursor is an aluminum oxide precursor and the second inorganic oxide precursor is a zinc oxide precursor.

In some embodiments, the first inorganic oxide precursor is a zinc precursor and the second inorganic oxide precursor is an aluminum oxide precursor.

In some embodiments, the aluminum oxide precursor is trimethylaluminum (TMA).

In some embodiments, the zinc oxide precursor is diethylzinc (DEZ).

In some embodiments, the inorganic oxide precursors are selected from the group consisting of a zinc oxide precursor, an aluminum oxide precursor and a silicon oxide precursor.

In some embodiments, each of the first and second inorganic oxide precursor is selected from DEZ and TMA and either: a) the first inorganic oxide precursor is TMA and the second inorganic oxide precursor is DEZ; or b) the first inorganic oxide precursor is DEZ and the second inorganic oxide precursor is TMA.

In some embodiments, each repeat of step (b) the first number is the same and selected from 1-10; and in each repeat of step (c) the second number is the same and selected from 1-10.

In some embodiments, the first number is 1 or 2.

In some embodiments, the second number is 2, 3, 4 or 5.

In some embodiments, the second number is between 1 and 5.

In some embodiments, the second number is between 1 and 5.

In some embodiments, steps (b)-(c) occur 1-40 times.

In some embodiments, each of steps (b1), (b3), (c1) and (c3) comprises: (i) introducing the vaporous or gaseous inorganic oxide precursor into the chamber, (ii) allowing a holding time to pass, and (iii) pumping the vaporous or gaseous inorganic oxide precursor of the chamber; and repeating steps (i)-(ii) at least once.

In some embodiments, some or all of the residual vaporous or gaseous first inorganic oxide precursor is pumped out of the reactor prior to step (b3).

In some embodiments, some or all of the residual vaporous or gaseous oxidant is pumped out of the reactor prior to step (c).

In some embodiments, some or all of the residual vaporous or gaseous second inorganic oxide precursor is pumped out of the reactor prior to step (c3).

In some embodiments, the first cycles and second cycles take place at a temperature between 25° C. and 60° C.

In some embodiments, the oxidant in step (b3) is water.

In some embodiments, the oxidant in step (c3) is water.

In some embodiments, step (a) further comprises agitating the particles.

In some embodiments, each pump-purge cycle comprises flowing the inert gas into the reactor chamber to a desired pressure and after a delay time pumping the inert gas out of the reactor until the pressure of the inert gas is below 1 torr and repeating the steps of flowing the inert gas into the reactor chamber to a desired pressure and after a delay time pumping the inert gas out of the reactor until the pressure of the inert gas is below 1 torr.

In some embodiments, a) the first and second inorganic acid precursors are selected from an aluminum oxide precursor and a zinc oxide precursor; b) the first and second inorganic acid precursors are selected from an aluminum oxide precursor and a silicone oxide precursor; or c) the first and second inorganic acid precursors are selected from an silicone oxide precursor and a zinc oxide precursor.

In some embodiments, a) the first and second inorganic acid precursors are selected from an aluminum oxide precursor and a zinc oxide precursor; or b) the first and second inorganic acid precursors are selected from an aluminum oxide precursor and a silicone oxide precursor.

In some embodiments, the method further comprises agitating the particles in the reactor throughout steps (a)-(d).

In some embodiments, the particles are not removed from the reactor during steps (a)-(d).

In some embodiments, method further comprises, after step (d):
(e) performing a third number of third cycles, wherein each third cycle comprises steps (e1)-(e4):
(e1) applying a vaporous or gaseous third inorganic oxide precursor to the particles in the reactor by pulsing the vaporous or gaseous third precursor into the reactor;
(e2) purging using an inert gas or performing one or more pump-purge cycles of the reactor using an inert gas;

(e3) applying a vaporous or gaseous oxidant to the particles in the reactor by pulsing the oxidant into the reactor;

(e4) purging using an inert gas or performing one or more pump-purge cycles of the reactor using an inert gas;
(f) performing a fourth number of fourth cycles, wherein each fourth cycle comprises steps (f1)-(f4):
(f1) applying a vaporous or gaseous fourth inorganic oxide precursor to the particles in the reactor by pulsing the vaporous or gaseous fourth precursor into the reactor;
(f2) purging using an inert gas or performing one or more pump-purge cycles of the reactor using an inert gas;
(f3) applying a vaporous or gaseous oxidant to the particles in the reactor by pulsing the oxidant into the reactor;
(f4) purging using an inert gas or performing one or more pump-purge cycles of the reactor using an inert gas; and
(g) repeating steps (e)-(f) at least once.

wherein, the third and fourth precursor are different; in each repeat of step (e) the third number is independently selected from 1-10 or 1-20; and in each repeat of step (f) the fourth number is independently selected from 1-10 or 1-20.

In some embodiments, the first and third inorganic oxide precursors are an aluminum oxide precursor and the second and fourth inorganic oxide precursors are a zinc oxide precursor.

In some embodiments, the first and third inorganic oxide precursors are an zinc oxide precursor and the second and fourth inorganic oxide precursors are an aluminum oxide precursor.

In some embodiments, the aluminum oxide precursor is trimethylaluminum (TMA).

In some embodiments, the zinc oxide precursor is diethylzinc (DEZ).

In some embodiments, the coating constitutes 1-20% wt/wt of the coated particles.

In some embodiments, steps (e)-(f) occur 1-40 times.

In some embodiments, the third number is 1 or 2 and the fourth number is between 1 and 10.

In some embodiments, the third number is 1 or 2.

In some embodiments, each in repeat of step (e) the third number is the same and selected from 1-10; and in each repeat of step (f) the fourth number is the same as selected from 1-10.

In one aspect, the disclosure is related to a coated particle prepared by the method described herein.

In some embodiments, the coated particle has a slower release compared to the uncoated particles.

In one aspect, the disclosure is related to a coated particle comprising a core comprising a polypeptide enclosed by an inorganic oxide coating, the coating comprising at least one layer composed of three elements (“three element inorganic oxide layer”), wherein the three elements are: a) aluminum, zinc and oxygen; b) aluminum, silicon and oxygen; or c) silicon, zinc and oxygen.

In some embodiments, the three element inorganic oxide layer is at least 2 nm thick.

In some embodiments, the three element inorganic oxide layer is 2-50 nm thick.

In some embodiments, the coated particle is 5-30% by weight inorganic oxide.

In some embodiments, the three element inorganic oxide layer is composed of: a) aluminum, zinc and oxygen and the ratio of aluminum to zinc varies in the layer; b) aluminum, silicone and oxygen and the ratio of aluminum to silicon varies in the layer; or c) silicon, zinc and oxygen and the ratio of silicon to zinc varies in the layer.

In some embodiments, the inorganic oxide coating further comprises at least one inorganic oxide coating layer composed of two elements (“two element inorganic oxide coating layer), wherein the two elements are: a) zinc and oxygen (zinc oxide); b) aluminum and oxygen (aluminum oxide); or c) silicon and oxygen (silicon oxide).

In some embodiments, the two element inorganic oxide layer is at least 1 nm thick.

In some embodiments, the two element inorganic oxide layer is 1-50 nm thick.

In some embodiments, the core consists of a polypeptide or a polypeptide and one or more pharmaceutically acceptable excipients.

In some embodiments, the core has a D50 on a volume average basis of 100 nm-30 micrometers.

In one aspect, the disclosure is related to a pharmaceutical composition comprising the coated particle described herein and a pharmaceutically acceptable excipient or carrier.

In one aspect, the disclosure is related to a pharmaceutical composition comprising the coated particle described herein and water.

In some embodiments, the method further comprises:
i. applying a vaporous or gaseous inorganic oxide precursor to the particles in the reactor by pulsing the vaporous or gaseous inorganic oxide precursor into the reactor;
ii. purging using an inert gas or performing one or more pump-purge cycles of the reactor using an inert gas;
iii. applying a vaporous or gaseous oxidant to the particles in the reactor by pulsing the oxidant into the reactor;
iv. purging using an inert gas or performing one or more pump-purge cycles of the reactor using an inert gas;
repeating steps i.-iv. at least once, wherein steps i.-iv. can take place: (1) between steps a) and b); 2) between steps d) and e); or after step g).

In some embodiments, the first inorganic oxide precursor is an aluminum oxide precursor, a zinc oxide precursor or a silicon oxide precursor.

In some embodiments, the aluminum oxide precursor is trimethylaluminum (TMA).

In some embodiments, the silicon oxide precursor is SiCl4, Tris (tertpentoxy) silanol, diisopropylamino silane (DIPAS) or 1,2-Bis (diisopropylamino) disilane (BDIPADS).

In one aspect, the disclosure is related to a method for preparing a polypeptide formulation, comprising (1) providing coated particles comprising a core comprising a polypeptide enclosed by a coating layer comprising an organic oxide selected from the group consisting of silicon oxide, titanium oxide, zinc oxide, aluminum oxide; and (2) mixing the coated particles with an aqueous composition, thereby creating a polypeptide formulation that comprises more than 100 mg/ml polypeptide.

In some embodiments, the aqueous composition is physiological saline.

In some embodiments, the core further comprises an excipient.

In some embodiments, the polypeptide formulation comprises more than 100 mg/ml coated particles.

In some embodiments, the coating layer is 0.1 nm-120 nm thick.

In some embodiments, the coating layer is 5 nm-15 nm thick.

In some embodiments, the coating layer is conformal and pin-hole free.

In some embodiments, the coated particle comprises 1-20% wt/wt inorganic oxide.

In some embodiments, the core consists of a polypeptide.

In some embodiments, the core comprises a polypeptide and one or more pharmaceutically acceptable excipients.

In some embodiments, the core has a D50 on a volume average basis of 100 nm-30 micrometers.

In some embodiments, the core has a median particle size, on a volume average basis, between 0.1 μm and 20 μm.

In some embodiments, the polypeptide is selected from the group consisting of a growth factor, an antigen, an antibody, and an antibody drug conjugate (ADC).

In some embodiments, the polypeptide is an antibody.

In some embodiments, the coating enhances the stability of the core.

In some embodiments, the coating reduces the viscosity of the formulation, as compared to a formulation containing uncoated particles.

In some embodiments, the coated particle has reduced surface charge compared to the uncoated core.

In some embodiments, the coated particle has enhanced hydrophilicity compared to the uncoated core.

In some embodiments, the coated particle has an improved flowability compared to the uncoated core.

In one aspect, the disclosure is related to a long acting injectable polypeptide formulation, comprising: a coated particle consisting of a core comprising a polypeptide, and an inorganic oxide coating layer enclosing the core, wherein the inorganic oxide coating layer comprises an inorganic oxide that comprises aluminum, zinc, silicon, and/or titanium, wherein the inorganic oxide coating layer slows the release rate of the long acting injectable polypeptide formulation. In one aspect, the disclosure is related to a polypeptide formulation, comprising: a coated particle consisting of a polypeptide-containing core comprising a polypeptide, and an inorganic oxide coating layer enclosing the polypeptide-containing core, wherein the inorganic oxide coating layer comprises an inorganic oxide that comprises aluminum, zinc, silicon, and/or titanium, wherein the inorganic oxide coating layer increases the stability of the polypeptide formulation.

In some embodiments, the coating reduces the amount of aggregation in the stable polypeptide formulation.

In some embodiments, the coating reduces the amount of degradation in the stable polypeptide formulation.

In one aspect, the disclosure provides a method of preparing a polypeptide formulation, the method comprising:
(a) loading particles comprising a polypeptide into a chamber of a reactor;
(b1) applying a vaporous or gaseous precursor to the particles in the reactor by pulsing the vaporous or gaseous precursor into the reactor;
(b2) performing one or more pump-purge cycles using an inert gas;
(b3) applying a vaporous or gaseous oxidant to the particles in the reactor by pulsing the oxidant into the reactor;
(b4) performing one or more pump-purge cycles using an inert gas;
(c) repeating steps (b1)-(b4) at least once to create a coated particle; and
(d) preparing a aqueous pharmaceutical formulation containing the coated particles. In some embodiments, each pump-purge cycle comprises flowing the inert gas into the reactor chamber to reach a desired pressure and after a delay time pumping the inert gas out of the reactor until the pressure of the inert gas is below 1 torr.

In some embodiments, the resulting high concentration polypeptide formulation comprises a coated particle consisting of a polypeptide-containing core comprising a polypeptide or a polypeptide and at least one excipient, wherein the coating layer is conformal, completely encloses the particles and comprises an inorganic oxide that comprises aluminum, zinc, silicon, and/or titanium.

In some embodiments, steps (b1)-(b4) take place at a temperature between 25° C. and 60° C. or between 25° C. and 50° C. or between 15° C. and 25° C.

In some embodiments, the particles are agitated during one or more of the steps. In some embodiments, one or both of steps (b2) and (b4) are replaced by a step in which comprises flowing an inert gas into the chamber or a step which comprising evacuating the chamber to below 1 torr.

In another aspect, the disclosure provides a method of preparing a polypeptide formulation containing coated polypeptide particles prepared using a so-called “supercycle” coating process that produces a mixed inorganic oxide layer. Thus, the disclose provides a method comprising:
(a) loading particles comprising a polypeptide and, optionally, an excipient into a chamber of a reactor;
(b) performing a first number of first cycles, wherein each first cycle comprises steps (b1)-(b4):
(b1) applying a vaporous or gaseous first inorganic oxide precursor to the particles in the reactor by pulsing the vaporous or gaseous first inorganic oxide precursor into the reactor;
(b2) purging using an inert gas or performing one or more pump-purge cycles of the reactor using an inert gas;
(b3) applying a vaporous or gaseous oxidant to the particles in the reactor by pulsing the oxidant into the reactor;
(b4) purging using an inert gas or performing one or more pump-purge cycles of the reactor using an inert gas;
(c) performing a second number of second cycles, wherein each second cycle comprises steps (c1)-(c4):
(c1) applying a vaporous or gaseous second inorganic oxide precursor to the particles in the reactor by pulsing the vaporous or gaseous second inorganic oxide precursor into the reactor;
(c2) purging using an inert gas or performing one or more pump-purge cycles of the reactor using an inert gas;
(c3) applying a vaporous or gaseous oxidant to the particles in the reactor by pulsing the oxidant into the reactor;
(c4) purging using an inert gas or performing one or more pump-purge cycles of the reactor using an inert gas; and
(d) repeating steps (b)-(c) at least once;
wherein, the first inorganic oxide precursor and second inorganic oxide precursor are different; in each repeat of step (b) the first number is independently selected from 1-10 or 1-20; and in each repeat of step (c) the second number is independently selected from 1-10 or 1-20.

In some embodiments, the precursor is an aluminum oxide precursor.

In some embodiments, the precursor is trimethylaluminium (TMA).

In some embodiments, the oxidant is water.

In some embodiments, the coating layer constitutes 1-20% wt/wt of the coated particle.

In some embodiments, the coating layer has a thickness in the range of 0.1 nm to 120 nm.

In some embodiments, the coating layer has a thickness in the range of 5 nm to 15 nm.

In some embodiments, the coated particles have enhanced hydrophilicity comparing to the uncoated polypeptide-containing core.

In some embodiments, the polypeptide is not degraded during the coating process.

The coating process can be performed at a low process temperature, e.g., at or below 25° C. In some embodiments, the operating temperature is 25° C. In some embodiments, the operating temperature is above 5° C., above 10° C., above 15° C., above 20° C., above 25° C., above 30° C., or above 35° C. In some embodiments, the operating temperature is below 5° C., below 10° C., below 15° C., below 20° C., below 25° C., below 30° C., or below 35° C. In some embodiments, the temperature is between 5° C. and 25° C., between 10° C. and 25° C. or between 15° C. and 25° C. In particular, the particles can remain or be maintained at such temperatures during all of the coating steps (e.g., inorganic precursor steps, oxidant steps, pump-purge steps). This can be achieved, for example, by having the oxidant gas, precursor gas and inert gas be injected into the chamber at such temperatures during the respective cycles. In addition, physical components of the chamber can remain or be maintained at such temperatures, e.g., using a cooling system, e.g., a thermoelectric cooler, if necessary.

In some embodiments, the weight percent of inorganic oxides constitutes more than 0.1%, more than 0.2%, more than 0.3%, more than 0.4%, more than 0.5%, more than 0.6%, more than 0.7%, more than 0.8%, more than 0.9%, more than 1%, more than 1.2%, more than 1.4%, more than 1.6%, more than 1.8%, more than 2%, more than 2.2%, more than 2.4%, more than 2.6%, more than 2.8%, more than 3%, more than 3.2%, more than 3.4%, more than 3.6%, more than 3.8%, more than 4%, more than 4.2%, more than 4.4%, more than 4.6%, more than 4.8%, more than 5%, more than 6%, more than 7%, more than 8%, more than 9%, more than 10%, more than 12%, more than 14%, more than 16%, more than 18%, or more than 20% wt/wt of the coated particles.

In some embodiments, weight percent of inorganic oxides in the coated particles is less than 0.1%, less than 0.2%, less than 0.3%, less than 0.4%, less than 0.5%, less than 0.6%, less than 0.7%, less than 0.8%, less than 0.9%, less than 1%, less than 1.2%, less than 1.4%, less than 1.6%, less than 1.8%, less than 2%, less than 2.2%, less than 2.4%, less than 2.6%, less than 2.8%, less than 3%, less than 3.2%, less than 3.4%, less than 3.6%, less than 3.8%, less than 4%, less than 4.2%, less than 4.4%, less than 4.6%, less than 4.8%, less than 5%, less than 6%, less than 7%, less than 8%, less than 9%, less than 10%, less than 12%, less than 14%, less than 16%, less than 18%, or less than 20% wt/wt of the coated particles. In some embodiments, the weight percent of inorganic oxides in the coated particles is 0.1%-20%, 0.5%-10%, 1%-10%, 1%-5%, 2%-5%, 1%-4%, 1%-3%, or 2%-4% wt/wt of the coated particles. In some embodiments, the amount of inorganic component constitutes about 1%-20% wt/wt of the coated particles.

In some embodiments, the coated particles have a reduced polypeptide release rate compared to uncoated particles. In some embodiments, the release rate of the polypeptide is at least more than 5%, more than 10%, more than 20%, more than 30%, more than 40%, more than 50%, more than 60%, more than 70%, more than 80%, more than 90%, more than 100%, more than 110%, more than 120%, more than 130%, more than 140%, more than 150%, more than 200%, more than 300%, more than 400%, more than 500%, or more than 600%, lower than the release rate of polypeptide in uncoated particles. In some embodiments, uncoated particles exhibited an immediate polypeptide release profile. In some embodiments, comparing to uncoated particles, the coated particles have less burst release of the polypeptide. In some embodiments, comparing to uncoated particles, the coated particles have a longer polypeptide release window.

In some embodiments, the coated particles have 30-90% polypeptide release in 30 minutes. In some embodiments, the coated particles have a slow polypeptide release rate. In some embodiments, the coated particles are dispersed in PBS.

In some embodiments, the coated particles have more than 10%, more than 20%, more than 30%, more than 40%, more than 50%, more than 60%, more than 70%, more than 80%, more than 90%, more than 95% polypeptide release in 30 minutes. In some embodiments, the coated particles have less than 10%, less than 20%, less than 30%, less than 40%, less than 50%, less than 60%, less than 70%, less than 80%, less than 90%, less than 95% polypeptide release in 30 minutes. In some embodiments, the coated particles have 100% polypeptide release in 30 minutes.

In some embodiments, comparing to uncoated particles, the coated particles have at least more than 10%, more than 20%, more than 30%, more than 40%, more than 50%, more than 60%, more than 70%, more than 80%, more than 90%, more than 95% slower polypeptide release.

In some embodiments, the uncoated particles have an immediate release (e.g., about 90-100% polypeptide release in 30 minutes). In some embodiments, the coated particles have a controlled release (e.g., about 1-10% polypeptide release in 30 minutes).

In some embodiments, the coated particles exhibit increased hydrophilicity comparing to the uncoated particles.

In some embodiments, the coated particles exhibit increased powder flowability (“FFc”) comparing to the uncoated particles.

In some embodiments, the coated particles exhibit increased bulk density comparing to the uncoated particles.

In some embodiments, the entirety of the coating layer has a thickness in the range of 0.1 nm to 100 nm, 0.1 nm to 50 nm, 0.1 nm to 10 nm, 0.1 to 5 nm, 1 nm to 50 nm, 1 nm to 10 nm, or 1 nm to 5 nm. In some embodiments, the entirety of the coating has a thickness of more than 0.1nm, more than 0.2 nm, more than 0.3 nm, more than 0.4 nm, more than 0.5 nm, more than 0.6 nm, more than 0.7 nm, more than 0.8 nm, more than 0.9 nm, more than 1 nm, more than 2 nm, more than 3 nm, more than 4 nm, more than 5 nm, more than 6 nm, more than 7 nm, more than 8 nm, more than 9 nm, more than 10 nm, more than 15 nm, more than 20 nm, more than 30 nm, more than 40 nm, more than 50 nm, or more than 100 nm. In some embodiments, the entirety of the coating has a thickness of less than 0.1 nm, less than 0.2 nm, less than 0.3 nm, less than 0.4 nm, less than 0.5 nm, less than 0.6 nm, less than 0.7 nm, less than 0.8 nm, less than 0.9 nm, less than 1 nm, less than 2 nm, less than 3 nm, less than 4 nm, less than 5 nm, less than 6 nm, less than 7 nm, less than 8 nm, less than 9 nm, less than 10 nm, less than 15 nm, less than 20 nm, less than 30 nm, less than 40 nm, less than 50 nm, or less than 100 nm. In some embodiments, the entirety of the coating has a thickness of between 10 nm and 50 nm. In some embodiments, the entirety of the coating has a thickness of between 10 nm and 200 nm, between 10 nm and 100 nm, between 10 nm and 50 nm, or between 25 nm and 50 nm. In some embodiments, the entirety of the coating has a thickness of 10-60 nm, 10-50 nm, 10-40 nm or 10-30 nm.

In some embodiments, an individual layer in a multi-layer coating has thickness in the range of 0.1 nm to 100 nm, 0.1 nm to 50 nm, 0.1 nm to 10 nm, 0.1 to 5 nm, 1 nm to 50 nm, 1 nm to 10 nm, or 1 nm to 5 nm. In some embodiments, the aluminum oxide layer has a thickness of more than 0.1 nm, more than 0.2 nm, more than 0.3 nm, more than 0.4 nm, more than 0.5 nm, more than 0.6 nm, more than 0.7 nm, more than 0.8 nm, more than 0.9 nm, more than 1 nm, more than 2 nm, more than 3 nm, more than 4 nm, more than 5 nm, more than 6 nm, more than 7 nm, more than 8 nm, more than 9 nm, more than 10 nm, more than 15 nm, more than 20 nm, more than 30 nm, more than 40 nm, more than 50 nm, or more than 100 nm. In some embodiments, the coating has a thickness of less than 0.1 nm, less than 0.2 nm, less than 0.3 nm, less than 0.4 nm, less than 0.5 nm, less than 0.6 nm, less than 0.7 nm, less than 0.8 nm, less than 0.9 nm, less than 1 nm, less than 2 nm, less than 3 nm, less than 4 nm, less than 5 nm, less than 6 nm, less than 7 nm, less than 8 nm, less than 9 nm, less than 10 nm, less than 15 nm, less than 20 nm, less than 30 nm, less than 40 nm, less than 50 nm, or less than 100 nm. In some embodiments, the coating has a thickness of between 1 nm and 30 nm. In some embodiments, the coating has a thickness of between 1 nm and 20 nm. In some embodiments, the coating has a thickness of 2-5 nm, 5-10 nm, or 10-20 nm.

In some embodiments, individual AZO, ASO, ZSO, ZnOx, AlOx, TiOx or SiOx layers can have a thickness of 1-10 nm, 5-10 nm, 5-20 nm or 10-20 nm.

As used herein, the terms “approximately” and “about,” as applied to one or more values of interest, refer to a value that is similar to a stated reference value. In certain embodiments, the term “approximately” or “about” refers to a range of values that fall within 25%, 20%, 19%, 18%, 17%, 16%, 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value). For example, when used in the context of an amount of a given compound in a composition, “about” may mean +/−10% of the recited value. For instance, a composition including about 100 ng/ml of a given compound may include 90˜110 ng/ml of the compound.

In some cases, polypeptide can have a molecular weight of 1-500 kD. The polypeptide can have a molecular weight of at least 1 kD, at least 2 kD, at least 3 kD, at least 4 kD, at least 5 kD, at least 6 kD, at least 7 kD, at least 8 kD, at least 9 kD, at least 10 kD, at least 20 kD, at least 30 kD, at least 40 kD, at least 50 kD, at least 100 kD, at least 150 kD, at least 200 kD, at least 250 kD, at least 300 kD, at least 350 kD, at least 400 kD, at least 450 kD, or at least 500 kD.

As used herein, the term polypeptide includes polypeptides of all sizes, including smaller polypeptides such a semaglutide (31 amino acids), longer polypeptides/proteins (for example polypeptide having more than 100 amino acids), and large multichain proteins (e.g., monoclonal antibodies). The term polypeptide includes molecules that are solely composed of one or more polypeptide chains as well as various polypeptide conjugates such as antibody-drug conjugates, polypeptide-polymer (e.g., polypeptide-PEG) conjugates, polypeptide-human serum albumin conjugates and other conjugates containing a polypeptide. Polypeptide drugs include polypeptides that can exert a therapeutic benefit, for example, an antibody, interferon, insulin and other therapeutic polypeptides.

Examples of polypeptides that can be present in coated particles include mammalian polypeptides, such as, e.g., growth hormone, including human growth hormone and bovine growth hormone; growth hormone releasing factor; parathyroid hormone; thyroid stimulating hormone; lipoproteins; α-1-antitrypsin; insulin A-chain; insulin B-chain; proinsulin; follicle stimulating hormone; calcitonin; luteinizing hormone; glucagon; clotting factors such as factor VIIIC, factor IX, tissue factor, and von Willebrands factor; anti-clotting factors such as Protein C; atrial natriuretic factor; lung surfactant; a plasminogen activator, such as urokinase or tissue-type plasminogen activator (t-PA, e.g., Activase®, TNKase®, Retevase®); bombazine; thrombin; tumor necrosis factor-a and-B; enkephalinase; RANTES (regulated on activation normally T-cell expressed and secreted); human macrophage inflammatory protein (MIP-1-α); serum albumin such as human serum albumin; mullerian-inhibiting substance; relaxin A-chain; relaxin B-chain; prorelaxin; mouse gonadotropin-associated peptide; DNase; inhibin; activin; vascular endothelial growth factor (VEGF); receptors for hormones or growth factors; an integrin; protein A or D; rheumatoid factors; a neurotrophic factor such as bone-derived neurotrophic factor (BDNF), neurotrophin-3, -4, -5, or -6 (NT-3, NT-4, NT-5, or NT-6), or a nerve growth factor such as NGF-β; platelet-derived growth factor (PDGF); fibroblast growth factor such as aFGF and bFGF; epidermal growth factor (EGF); transforming growth factor (TGF) such as TGF-α and TGF-β, including TGF-β1, TGF-β2, TGF-β3, TGF-β4, or TGF-β5; insulin-like growth factor-I and -II (IGF-I and IGF-II); des(1-3)-IGF-I (brain IGF-I); insulin-like growth factor binding proteins; CD proteins such as CD3, CD4, CD8, CD19 and CD20; erythropoietin (EPO); thrombopoietin (TPO); osteoinductive factors; immunotoxins; a bone morphogenetic protein (BMP); an interferon such as interferon-α, -β, and -γ; colony stimulating factors (CSFs), e.g., M-CSF, GM-CSF, and G-CSF; interleukins (ILs), e.g., IL-1 to IL-10; superoxide dismutase; T-cell receptors; surface membrane proteins; decay accelerating factor (DAF); a viral antigen such as, for example, a portion of the AIDS envelope; transport polypeptides; homing receptors; addressins; regulatory proteins; immunoadhesins; antibodies; and biologically active fragments or variants of any of the above-listed polypeptides.

In some embodiments, the polypeptide is an antibody. Exemplary molecular targets for antibodies encompassed by the present invention include IgE, the CD proteins CD3, CD4, CD8, CD19, CD20, CD34 and CD40; members of the HER receptor family such as EGF receptor, HER2, HER3 or HER4 receptor; 2c4, 4D5, PSCA, LDP-2, cell adhesion molecules such as LFA-1, Mac1, p150, 95, VLA-4, ICAM-1, VCAM and αv/β3 integrin including the α- and β-subunits thereof (e.g., anti-CD1la, anti-CD18 or anti-CD11b antibodies); growth factors such as VEGF; blood group antigens; flk2/flt3 receptor; obesity (OB) receptor; mpl receptor, CTLA-4, and Protein C.

As used herein, the term “antibody” includes monoclonal antibodies (including full length antibodies which have an immunoglobulin Fc region), antibody compositions with polyepitopic specificity, multispecific antibodies (e.g., bispecific antibodies, diabodies, and single-chain molecules, as well as antibody fragments (e.g., Fab, F(ab′)₂, and Fv). In some embodiments, the term “immunoglobulin” (Ig) is used interchangeably with “antibody” herein. The coating can increase the stability of the polypeptide such that it retains its physical and chemical stability and integrity upon storage for a longer period of time. Stability can be measured at a selected temperature for a selected time period. For rapid screening, the formulation may be kept at 40° C. for 2 weeks to 1 month, at which time stability is measured. Where the formulation is to be stored at 2-8° C., it is desirable for the formulation to be stable at 30° C. or 40° C. for at least 1 month and/or stable at 2-8° C. for at least 2 years. Where the formulation is to be stored at 30° C., it is desirable for the formulation to be stable for at least 1 or 2 years at 30° C. and/or stable at 40° C. for at least 6 months. For example, the extent of aggregation during storage can be used as an indicator of polypeptide stability. Thus, a formulation with increased stability can be one wherein less than about 10% and preferably less than about 5% of the polypeptide are present as an aggregate in the formulation after storage for a desired period of time. In some cases, stability can be measured by the amount of degradation (e.g., measured by HPLC-SEC). In some cases, stability is measured by storage at 40° C., 75% relative humidity for 6 months.

As used herein, the term “viscosity” may refer to “kinematic viscosity” or “absolute viscosity.” “Kinematic viscosity” is a measure of the resistive flow of a fluid under the influence of gravity. When two fluids of equal volume are placed in identical capillary viscometers and allowed to flow by gravity, a viscous fluid takes longer than a less viscous fluid to flow through the capillary. If one fluid takes 200 seconds to complete its flow and another fluid takes 400 seconds, the second fluid is twice as viscous as the first on a kinematic viscosity scale. “Absolute viscosity”, sometimes called dynamic or simple viscosity, is the product of kinematic viscosity and fluid density: Absolute Viscosity=Kinematic Viscosity×Density. The dimension of kinematic viscosity is L2/T where L is a length and T is a time. Commonly, kinematic viscosity is expressed in centistokes (cSt). The SI unit of kinematic viscosity is mm2/s, which is 1 cSt. Absolute viscosity is expressed in units of centipoise (cP). The SI unit of absolute viscosity is the milliPascal-second (mPa-s), where 1 cP=1 mPa-s. The viscosity can be measured with a rheology instrument.

Unless otherwise defined, all technical and scientific terms used herein have the same meaning as commonly understood by one of ordinary skill in the art to which this invention belongs. Methods and materials are described herein for use in the present invention; other, suitable methods and materials known in the art can also be used. The materials, methods, and examples are illustrative only and not intended to be limiting. All publications, patent applications, patents, sequences, database entries, and other references mentioned herein are incorporated by reference in their entirety. In case of conflict, the present specification, including definitions, will control.

### BRIEF DESCRIPTION OF THE DRAWINGS

FIG. 1 depicts a schematic illustration of an exemplary reactor system.

FIG. 2 depicts a schematic illustration of an exemplary supercycle process. The exemplary supercycle process contains n number of AlOx cycles and m number of ZnOx cycles. The AlOx cycle includes applying a vaporous or gaseous aluminum precursor (e.g., trimethylaluminum or TMA); performing one or more pump-purge cycles of the reactor using an inert gas; applying a vaporous or gaseous oxidant (e.g., H₂O) to the particles in the reactor by pulsing the oxidant into the reactor; and performing one or more pump-purge cycles of the reactor using an inert gas. The ZnOx cycle includes applying a vaporous or gaseous zinc precursor (e.g., diethylzinc or DEZ); performing one or more pump-purge cycles of the reactor using an inert gas; applying a vaporous or gaseous oxidant (e.g., H₂O) to the particles in the reactor by pulsing the oxidant into the reactor; and performing one or more pump-purge cycles of the reactor using an inert gas.

FIG. 3 depicts a schematic illustration of an exemplary cycle in a supercycle process. The cycle contains applying a vaporous or gaseous precursor (e.g., TMA or DEZ); performing one or more pump-purge cycles of the reactor using an inert gas; applying a vaporous or gaseous oxidant (e.g., H₂O) to the particles in the reactor by pulsing the oxidant into the reactor; and performing one or more pump-purge cycles of the reactor using an inert gas.

FIG. 4 depicts a schematic illustration of particle coated with multiple layers. In this example, an AlOx layer is applied using conventional cycles, then an AZO layer is applied using supercycles. Finally a ZnOx layer is applied using conventional cycles. The Al/Zn ratio in the AZO layer can be adjusted by varying the numbers of AlOx cycles and ZnOx cycles. The overall coating thickness and the coating wt % can be adjusted by varying the number of conventional cycles and the number of supercycles.

FIGS. 5A-5B show a comparison of traditional long acting injectable manufacturing processes based on microspheres (FIG. 5A) and the coating methods described herein (FIG. 5B). The microsphere-based method comprises at least six steps and requires large volumes of solvents (e.g., water and oil). By comparison, the coating methods described herein simplifies the process and reduces solid, liquid, and energy waste.

FIG. 6 shows an exemplary coating process that includes a first half cycle (precursor dosing and purge) and a second half cycle (co-reactant exposure and purge).

FIGS. 7A-7B show additional exemplary reactor designs.

FIG. 8A shows Scanning Electron Microscopy (SEM) and energy dispersive X-ray spectroscopy (EDS) images of an exemplary coated polypeptide particle encapsulated by an aluminum oxide coating.

FIG. 8B shows SEM and EDS images of an exemplary coated polypeptide particle encapsulated by a zinc oxide coating.

FIG. 9A shows a SEM image of an exemplary lyophilized polypeptide before any coating is applied.

FIG. 9B shows a cross-sectional Transmission Electron Microscopy (TEM) and Focused Ion Beam (FIB) image of an exemplary coated polypeptide particle encapsulated by an aluminum oxide coating.

FIG. 9C shows a SEM image of an exemplary spray-dried polypeptide before any coating is applied.

FIG. 9D shows a cross-sectional Transmission Electron Microscopy (TEM) and Focused Ion Beam (FIB) image of an exemplary coated polypeptide particle encapsulated by an aluminum oxide coating.

### DETAILED DESCRIPTION

This disclosure is related to polypeptide formulations comprising coated polypeptide-containing particles. The coated particles have an inorganic oxide coating layer enclosing a core that includes a polypeptide and, optionally, one or more excipients. The coating is applied by vapor phase deposition. Aqueous formulations containing the coated particles can have a high polypeptide content without having an undesirably high viscosity, making them suitable for injection. The coated particles can have a reduced surface charge as compared to the uncoated particles and/or may have enhanced hydrophilicity as compared to the uncoated particles. To create a high polypeptide content formulation, the coated particles may be mixed with an aqueous composition (e.g., physiological saline) to form a suspension. The coated particles can have improved stability as compared with uncoated particles. The coated particles can also have a slower release rate as compared with uncoated particles.

The polypeptide compositions can be formulated in any suitable manner known in the art.

Pharmaceutical compositions are formulated to be compatible with their intended route of administration (e.g., subcutaneous). The compositions can include a sterile diluent (e.g., sterile water or saline), a fixed oil, polyethylene glycol, glycerine, propylene glycol or other synthetic solvents, antibacterial or antifungal agents (e.g., benzyl alcohol or methyl parabens, chlorobutanol, phenol, ascorbic acid, and thimerosal), antioxidants (e.g., ascorbic acid and sodium bisulfite), chelating agents (e.g., ethylenediaminetetraacetic acid), buffers (e.g., acetates, citrates, and phosphates), and isotonic agents (e.g., sugars (e.g., dextrose), polyalcohols (e.g., mannitol or sorbitol), and salts (e.g., sodium chloride)), or any combination thereof. Liposomal suspensions can also be used as pharmaceutically acceptable carriers (see, e.g., U.S. Pat. No. 4,522,811). Preparations of the compositions can be formulated and enclosed in ampules, disposable syringes, or multiple dose vials. Where required (as in, for example, injectable formulations), proper fluidity can be maintained by, for example, the use of a coating (e.g., lecithin) or a surfactant. Controlled release can be achieved by implants and microencapsulated delivery systems, which can include biodegradable, biocompatible polymers (e.g., ethylene vinyl acetate, polyanhydrides, polyglycolic acid, collagen, polyorthoesters, and polylactic acid).

Pharmaceutically acceptable carriers, adjuvants and vehicles that can be used in the pharmaceutical compositions of the present disclosure include ion exchangers, alumina, aluminum stearate, lecithin, serum polypeptides (e.g., human serum albumin), buffer substances (e.g., phosphates, glycine), sorbic acid, potassium sorbate, partial glyceride mixtures of saturated vegetable fatty acids, water, salts or electrolytes (e.g., protamine sulfate, disodium hydrogen phosphate, potassium hydrogen phosphate, sodium chloride, and zinc salts), colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol, and wool fat.

### High Concentration Polypeptide Formulations

Pharmaceutical development challenges for high concentration polypeptide formulations include: high solution viscosity and/or physical instability, aggregation and/or limited solubility, leading to difficulties during large-scale manufacturing, long-term storage, and delivery/administration to patients. High concentration polypeptide formulations can have poor syringability. Moreover, the presence of sugars and other excipients can increase intermolecular the interactions that increase viscosity. The force required to manipulate viscous formulations can cause polypeptide denaturation.

The high concentration (i.e., high polypeptide content) polypeptide formulation may comprise more than about 100 mg/ml, about 120 mg/ml, about 150 mg/ml, about 200 mg/ml, about 500 mg/ml, or about 1000 mg/ml coated particles. The high concentration polypeptide formulation may comprise less than about 100 mg/ml, about 120 mg/ml, about 150 mg/ml, about 200 mg/ml, about 500 mg/ml, or about 1000 mg/ml coated particles. The high concentration polypeptide formulation may comprise more than about 100 mg/ml coated particles.

The compositions or formulations can contain the coated particles described herein in the range of 0.001% to 100% wt/wt (e.g., 0.1-95%, 20-80%, or 75-85% wt/cwt) with the balance made up from the suitable pharmaceutically acceptable excipients.

The high concentration polypeptide formulation can have a reduced viscosity, as compared to a similar formulation prepared with uncoated particles or a similar formulation having the same polypeptide content. The coating described herein can help reduce the viscosity of the high concentration polypeptide formulation by reducing release of the polypeptide from the particles.

The high concentration polypeptide formulation can have a reduced amount of polypeptide aggregation after storage. The amount of polypeptide aggregation can be assessed with size exclusion chromatography (SEC) and/or HPLC. Comparing to uncoated particles, the polypeptide aggregation in the coated particles may be reduced by more than 5%, more than 10%, more than 20%, more than 30%, more than 40%, more than 50%, more than 60%, more than 70%, more than 80%, more than 90%, more than 100%, more than 110%, more than 120%, more than 130%, more than 140%, more than 150%, more than 200%, more than 300%, more than 400%, more than 500%, or more than 600%.

The coating can simplify the formulation process or other aspects of drug product manufacturing. For example, the coating can eliminate the need to include additional excipients (e.g., detergents) in the final formulation. The coating may eliminate the need to include additional viscosity reducing agents in the final formulation.

### Long Acting Injectable (LAI) Polypeptide Formulations

Traditional non-LAI polypeptide products suffer from frequent injections and patient compliance issues, especially for chronic diseases. In one aspect, the disclosure provides a long acting injectable (LAI) formulation with a controlled release profile. The coated polypeptide particles described herein may have a slower polypeptide release rate as compared with uncoated polypeptide particles. Further, the coated polypeptide particles may have an increased stability as compared with uncoated polypeptide particles. In one aspect, the disclosure provides a method to control polypeptide release from a polypeptide containing particle by by coating the particle with an inorganic oxide.

The coating can lead to lower burst release and stable drug levels in plasma for a tunable extended period. The polypeptide (e.g., antibody) release from the coated particles can be assessed by an in vitro release over time analysis. For example, the polypeptide release can be assessed by HPLC analysis. The release may be assessed in methanol. The release may be assessed in a sodium phosphate buffer solution (PBS) (e.g., pH 7.2, with or without surfactant) at 37° C., with a stirring of 100 revolutions per minute (RPM), for more than 1 minute, more than 2 minutes, more than 5 minutes, more than 10 minutes, more than 20 minutes, more than 30 minutes, more than 40 minutes, more than 50 minutes, more than 60 minutes, more than 120 minutes, more than 3 hours, more than 4 hours, more than 5 hours, more than 6 hours, more than 7 hours, more than 8 hours, more than 12 hours, more than 16 hours, more than 24 hours. The coated particles may have a reduced release rate compared to uncoated particles. The release rate of the coated particles may be at least more than 5%, more than 10%, more than 20%, more than 30%, more than 40%, more than 50%, more than 60%, more than 70%, more than 80%, more than 90%, more than 100%, more than 110%, more than 120%, more than 130%, more than 140%, more than 150%, more than 200%, more than 300%, more than 400%, more than 500%, or more than 600%, slower than the release rate of uncoated particles. Uncoated particles may exhibit an immediate release profile (burst release). The coated particles may have 30-90% polypeptide (e.g., antibody) release in 30 minutes.

The coated particles may have less than 10%, less than 20%, less than 30%, less than 40%, less than 50%, less than 60%, less than 70%, less than 80%, less than 90%, less than 95% polypeptide (e.g., antibody) release in 30 minutes. The coated particles may have 100% polypeptide (e.g., antibody) release in 30 minutes. Comparing to uncoated particles, the coated particles may have at least less than 10%, less than 20%, less than 30%, less than 40%, less than 50%, less than 60%, less than 70%, less than 80%, less than 90%, less than 95% polypeptide (e.g., antibody) release. The uncoated particles may have an immediate release (e.g., about 90-100% polypeptide release in 30 minutes). The coated particles may have a controlled release (e.g., about 1-10% release in 30 minutes).

### Stable Polypeptide Formulations

Traditional polypeptide products can suffer from poor stability and require cold chain storage and transportation. In one aspect, the disclosure provides polypeptide formulations with increased stability, reducing the need for cold chain storage and transportation. The polypeptide within the coated particles can have improved stability as compared with the polypeptide in uncoated particles. Without being bound by theory, the coating layer can act as a physical/chemical barrier to reduce exposure of the coated particles to water or oxidants (e.g., oxygen) in the environment. Also, the coating layer may act as a physical barrier to protect the coated particles from mechanical forces (e.g., shear force) during manufacturing and handling. Without being bound by theory, the coating layer may also reduce the mobility of the protein and/or reduce undesirable protein-protein interactions.

The polypeptide formulation can have a reduced amount of polypeptide aggregation after storage. The amount of polypeptide aggregation can be assessed with size exclusion chromatography (SEC) and/or HPLC. Comparing to uncoated particles, the polypeptide aggregation in the coated particles may be reduced by more than 5%, more than 10%, more than 20%, more than 30%, more than 40%, more than 50%, more than 60%, more than 70%, more than 80%, more than 90%, more than 100%, more than 110%, more than 120%, more than 130%, more than 140%, more than 150%, more than 200%, more than 300%, more than 400%, more than 500%, or more than 600%.

The polypeptide formulation can have a reduced amount of degradation after storage. The amount of polypeptide degradation can be assessed with size exclusion chromatography (SEC) and/or HPLC. Comparing to uncoated particles, the polypeptide aggregation in the coated particles may be reduced by more than 5%, more than 10%, more than 20%, more than 30%, more than 40%, more than 50%, more than 60%, more than 70%, more than 80%, more than 90%, more than 100%, more than 110%, more than 120%, more than 130%, more than 140%, more than 150%, more than 200%, more than 300%, more than 400%, more than 500%, or more than 600%.

### Uncoated Particles

The uncoated particles include a be polypeptide that can be entirely composed of one or more polypeptide chains or can be composed of a polypeptide conjugate, e.g., a polypeptide conjugated to a small molecule drug, a carbohydrate, a polymer or another polypeptide. carThe uncoated particles may further contain one or more pharmaceutically acceptable excipients, for example a sugar or other agent that stabilizes the polypeptide.

The uncoated particles may contain at least 10%, 20%, 30%, 40%, or 50% wt/wt polypeptides. The uncoated particles may contain less than 10%, 20%, 30%, 40%, or 50% wt/wt polypeptides. The uncoated particles may contain at least 60%, 70%, 80%, 90%, 99% or 100% wt/wt polypeptides. The uncoated particles may contain at least 10%, 20%, 30%, 40%, or 50% wt/wt antibodies. The uncoated particles may contain less than 10%, 20%, 30%, 40%, or 50% wt/wt antibodies. The uncoated particles may contain at least 60%, 70%, 80%, 90%, 99% or 100% wt/wt antibodies.

The uncoated particles may have a D10 of less than 0.1 μm, less than 0.2 μm, less than 0.5 μm, less than 1 μm, less than 2 μm, less than 5 μm, less than 10 μm, less than 20 μm, or less than 50 μm, on a volume average basis. The uncoated particles may have a D10 of more than 0.1 μm, more than 0.2 μm, more than 0.5 μm, more than 1 μm, more than 2 μm, more than 5 μm, more than 10 μm, more than 20 μm, or more than 50 μm, on a volume average basis. The uncoated particles may have a D10 of 0.1 μm to 200 μm, 0.1 μm to 1 μm, 0.1 μm to 10 μm, or 0.1 μm to 50 μm on a volume average basis. The uncoated particles may have a D10 of about 2 μm on a volume average basis.

The uncoated particles may have a D50 of less than 0.1 μm, less than 0.2 μm, less than 0.5 μm, less than 1 μm, less than 2 μm, less than 5 μm, less than 10 μm, less than 20 μm, or less than 50 μm, on a volume average basis. The uncoated particles may have a D50 of more than 0.1 μm, more than 0.2 μm, more than 0.5 μm, more than 1 μm, more than 2 μm, more than 5 μm, more than 10 μm, more than 20 μm, or more than 50 μm, on a volume average basis. The uncoated particles may have a D50 of 0.1 μm to 200 μm, 0.1 μm to 1 μm, 0.1 μm to 10 μm, 1 μm to 20 μm, or 0.1 μm to 50 μm on a volume average basis. The uncoated particles may have a D50 of about 5-10 μm on a volume average basis. In some cases, the uncoated particles may have a

D50 of about 1-25 μm.

The uncoated particles may have a D90 of less than 0.1 μm, less than 0.2 μm, less than 0.5 μm, less than 1 μm, less than 2 μm, less than 5 μm, less than 10 μm, less than 20 μm, or less than 50 μm, on a volume average basis. The uncoated particles may have a D90 of more than 0.1 μm, more than 0.2 μm, more than 0.5 μm, more than 1 μm, more than 2 μm, more than 5 μm, more than 10 μm, more than 20 μm, or more than 50 μm, on a volume average basis. The uncoated particles may have a D90 of 200 μm to 2000 μm on a volume average basis. The uncoated particles may have a D50 of 0.1 μm to 200 μm, 0.1 μm to 1 μm, 0.1 μm to 10 μm, or 0.1 μm to 50 μm on a volume average basis. The uncoated particles may have a D90 of about 9.2 μm on a volume average basis.

The uncoated particles may be roughly spherical. The uncoated particles may be flake-shaped. The uncoated particles may have an aspect ratio (maximum dimension/minimum dimension) of between 0.5 and 1.5, between 5 and 10, between 5 and 20, between 5 and 50, or greater than 40.

In various embodiments: the uncoated particles have an aspect ratio of between 5 and 10, and 20, 5 and 50 or greater than 40; the particles have a D50 of 0.1 μm to 100 μm on a volume average basis; the particles have a D50 of 0.1 μm to 20 μm on a volume average basis; the particles have a D90 of 0.1 μm to 100 μm on a volume average basis; the particles have a D90 of less than 30 μm on a volume average basis; the particles are dendrites having at least one branch; the particles have an average of at least 3 ends; the uncoated particles have an average of at least 2, 3, 4 or 5 branches; the specific surface area of the particles is: greater than 2 m²/g, greater than 4 m²/g, greater than 6 m²/g, greater than 8 m²/g, or between 2 or 4 and 8 m²/g.

The uncoated particles can be lyophilized polypeptide particles. The uncoated particles can be prepared by freeze drying (lyophilization) or spray drying. Freeze drying temperatures are generally below 0° C. during the primary drying stage. During the process the ice crystal structure of the specimen is manipulated to remove unbound water and moisture. For the secondary drying stage temperatures of between 20° C. and 30° C. are usually used. This process removes excess bound water and is more energy intensive. The secondary stage is carried out in a low atmospheric pressure environment, which forces ice to transform into water vapor. Depending on the sample type, the lyophilization cycle can take anywhere from a matter of hours to several days.

In some cases, the uncoated polypeptide particles can be pre pared by spray drying. Unlike lyophilization, spray drying transforms a liquid sample into a dry powder in a single step. After the sample is atomized, miniscule droplets are sent into a chamber and dried using hot gas. A cyclone can be used to collect dry particles, which form the final spray dried product. While useful for some applications, spray drying relies on high processing temperatures and force.

Lyophilization The uncoated particles described herein may be prepared by lyophilization. A “pre-lyophilized solution” may be produced by mixing the polypeptide with a lyoprotectant. As used herein, a “lyoprotectant” is a molecule which, when combined with a polypeptide of interest, significantly prevents or reduces chemical and/or physical instability of the polypeptide upon lyophilization and subsequent storage. Exemplary lyoprotectants include sugars and their corresponding sugar alcohols; an amino acid such as monosodium glutamate or histidine; a methylamine such as betaine; a lyotropic salt such as magnesium sulfate; a polyol such as trihydric or higher molecular weight sugar alcohols, e.g. glycerin, dextran, erythritol, glycerol, arabitol, xylitol, sorbitol, and mannitol; propylene glycol; polyethylene glycol; Pluronics®; and combinations thereof. Additional exemplary lyoprotectants include glycerin and gelatin, and the sugars mellibiose, melezitose, raffinose, mannotriose and stachyose. Examples of reducing sugars include glucose, maltose, lactose, maltulose, iso-maltulose and lactulose. Examples of non-reducing sugars include non-reducing glycosides of polyhydroxy compounds selected from sugar alcohols and other straight chain polyalcohols. Preferred sugar alcohols are monoglycosides, especially those compounds obtained by reduction of disaccharides such as lactose, maltose, lactulose and maltulose. The glycosidic side group can be either glucosidic or galactosidic. Additional examples of sugar alcohols are glucitol, maltitol, lactitol and iso-maltulose. The preferred lyoprotectant are the non-reducing sugars trehalose or sucrose.

The polypeptide to be formulated may be present in a pre-lyophilized solution. A lyoprotectant may be added to the pre-lyophilized solution. The amount of lyoprotectant must not be too low such that an unacceptable amount of degradation/aggregation of the polypeptide occurs upon lyophilization. Exemplary lyoprotectant concentrations in the pre-lyophilized solution are from about 10 mM to about 400 mM, alternatively from about 30 mM to about 300 mM, alternatively from about 50 mM to about 100 mM. Exemplary lyoprotectants include sugars and sugar alcohols such as sucrose, mannose, trehalose, glucose, sorbitol, mannitol.

The ratio of polypeptide to lyoprotectant can vary for each particular polypeptide or polypeptide and lyoprotectant combination. In the case of a polypeptide as the polypeptide of choice and a sugar (e.g., sucrose or trehalose) as the lyoprotectant for generating an isotonic reconstituted formulation with a high polypeptide concentration, the molar ratio of lyoprotectant to polypeptide may be from about 100 to about 1500 moles lyoprotectant to 1 mole polypeptide, and preferably from about 200 to about 1000 moles of lyoprotectant to 1 mole polypeptide, for example from about 200 to about 600 moles of lyoprotectant to 1 mole polypeptide.

It may be desirable to add a surfactant to the pre-lyophilized solution. Exemplary surfactants include nonionic surfactants such as polysorbates (e.g. polysorbates 20 or 80); polyoxamers (e.g. poloxamer 188); Triton; sodium octyl glycoside; lauryl-, myristyl-, linoleyl-, or stearyl-sulfobetaine; lauryl-, myristyl-, linoleyl-or stearyl-sarcosine; linoleyl-, myristyl-, or cetyl-betaine; lauroamidopropyl-, cocamidopropyl-, linolcamidopropyl-, myristamidopropyl-, palmidopropyl-, or isostearamidopropyl-betaine (e.g. lauroamidopropyl); myristamidopropyl-, palmidopropyl-, or isostearamidopropyl-dime thylamine; sodium methyl cocoyl-, or disodium methyl oleyl-taurate; and the MONAQUA™ series (Mona Industries, Inc., Paterson, N.J.), polyethyl glycol, polypropyl glycol, and copolymers of ethylene and propylene glycol (e.g. Pluronics, PF68 etc.).

A mixture of the lyoprotectant (such as sucrose or trehalose) and a bulking agent (e.g. mannitol or glycine) may be used in the preparation of the pre-lyophilized solution. The bulking agent may allow for the production of a uniform lyophilized cake without excessive pockets therein etc. Other pharmaceutically acceptable carriers, excipients or stabilizers such as those described in Remington's Pharmaceutical Sciences 16th edition, Osol, A. Ed. (1980) may be included in the pre-lyophilized solution provided that they do not adversely affect the desired characteristics of the formulation. Acceptable carriers, excipients or stabilizers are nontoxic to recipients at the dosages and concentrations employed and include; additional buffering agents; preservatives; co-solvents; antioxidants including ascorbic acid and methionine; chelating agents such as EDTA; metal complexes (e.g. Zn-polypeptide complexes); biodegradable polymers such as polyesters; and/or salt-forming counterions such as sodium.

The pre-lyophilized may also contain more than one polypeptide as necessary for the particular indication being treated, preferably those with complementary activities that do not adversely affect the other polypeptide. For example, it may be desirable to provide two or more antibodies which bind to the desired target (e.g., receptor or antigen) in a single formulation. Such polypeptides are suitably present in combination in amounts that are effective for the purpose intended.

The formulations to be used for in vivo administration must be sterile. This is readily accomplished by filtration through sterile filtration membranes, prior to, or following, lyophilization and reconstitution. Alternatively, sterility of the entire mixture may be accomplished by autoclaving the ingredients, except for polypeptide, at about 120° C. for about 30 minutes, for example.

After the polypeptide, optional lyoprotectant and other optional components are mixed together, the pre-lyophilized solution is lyophilized in a freeze-dryer. Freeze-drying is accomplished by freezing the formulation and subsequently subliming ice from the frozen content at a temperature suitable for primary drying. Under this condition, the product temperature is below the eutectic point or the collapse temperature of the formulation. Typically, the shelf temperature for the primary drying will range from about −30 to 25° C. (provided the product remains frozen during primary drying) at a suitable pressure, ranging typically from about 50 to 250 mTorr. The formulation, size and type of the container holding the sample (e.g., glass vial) and the volume of liquid will mainly dictate the time required for drying, which can range from a few hours to several days (e.g. 40-60 hrs). Optionally, a secondary drying stage may also be performed depending upon the desired residual moisture level in the product. The temperature at which the secondary drying is carried out ranges from about 0-40° C., depending primarily on the type and size of container and the type of polypeptide employed. For example, the shelf temperature throughout the entire water removal phase of lyophilization may be from about 15-30° C. (e.g., about 20° C.). The time and pressure required for secondary drying will be that which produces a suitable lyophilized cake, dependent, e.g., on the temperature and other parameters. The secondary drying time is dictated by the desired residual moisture level in the product and typically takes at least about 5 hours (e.g. 10-15 hours). The pressure may be the same as that employed during the primary drying step.

After lyophilization, additional processing (e.g., milling) may be applied to reduce the particle size of the uncoated particles and to make the particle size relatively more uniform before the coating is applied. Without being bound by theory, a relatively uniform particle size can help facilitate the coating process.

### Vapor Phase Deposition

In one aspect, coatings are applied to the uncoated particles by vapor phase deposition using a precursor molecule (e.g., an inorganic oxide precursor) and an oxidant (e.g., ozone or water vapor). Vapor phase deposition of inorganic oxides (e.g., metal oxides or metalloid oxides) is sometimes referred to as atomic layer deposition (ALD). However, depending on a number of factors, including the surface being coated, each cycle of the deposition reaction does not necessarily deposit one atomic layer on the entire surface.

The coating method described herein is a solvent-free dry process which deposits uniform and conformal coatings of nanometer thickness at a low temperature with high precision. The coating can be tailored in terms of coating material, coating thickness, composition, and morphology.

### Reactor System

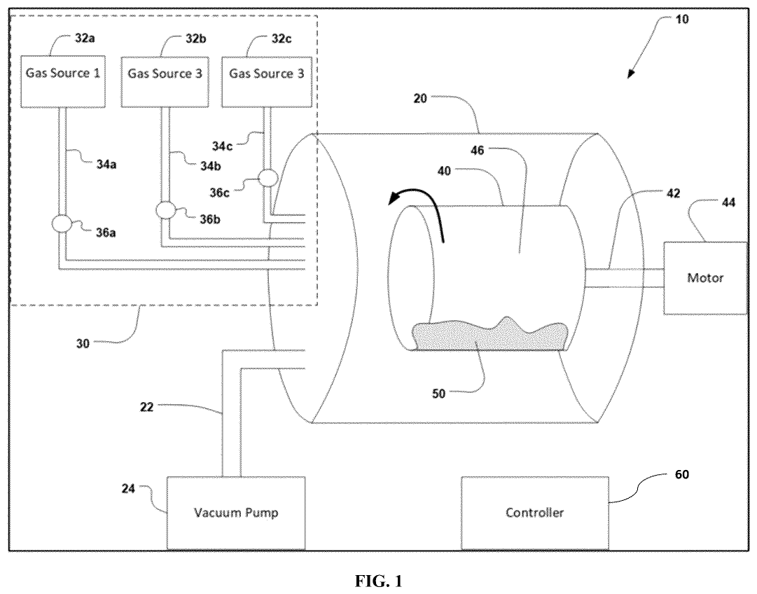

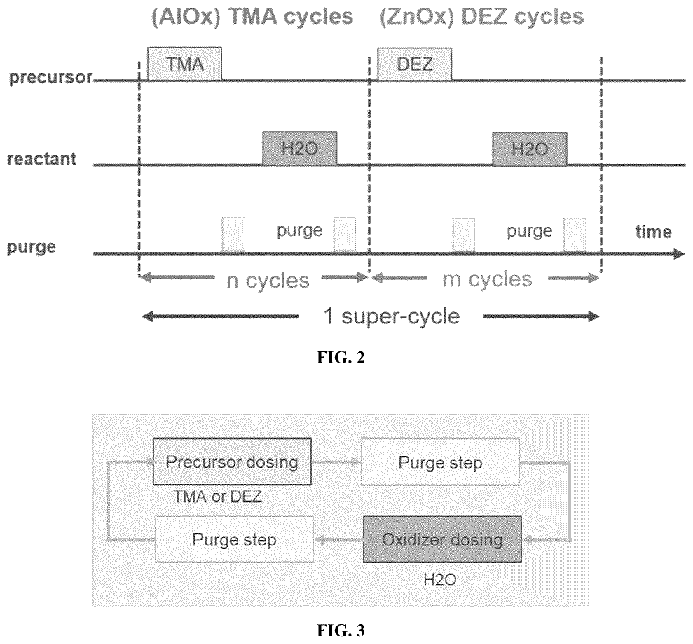

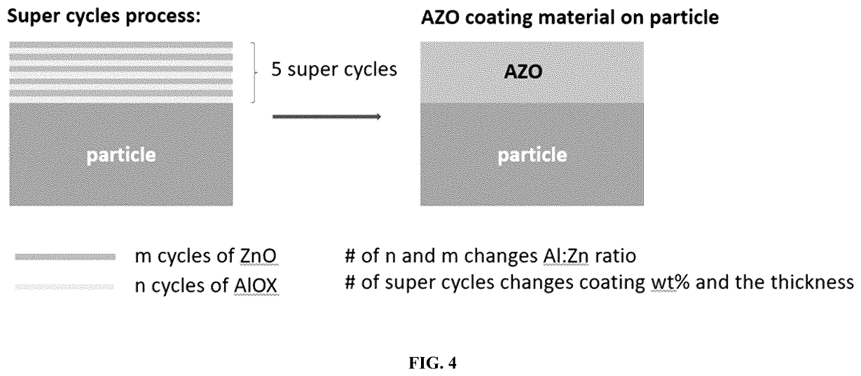

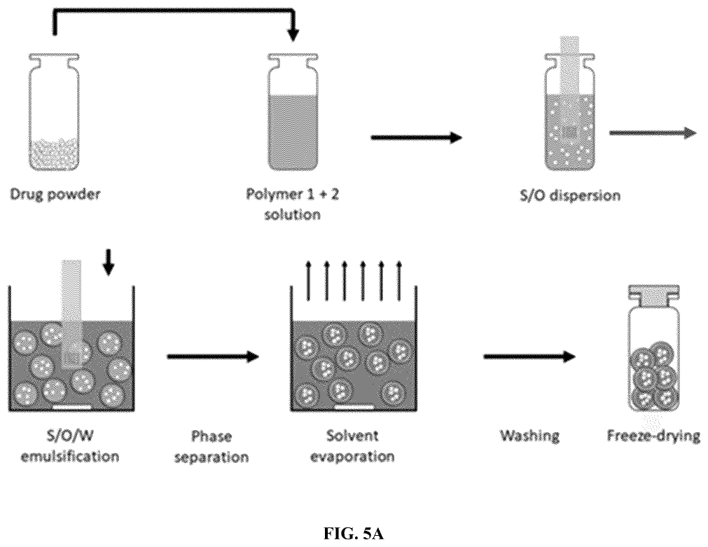

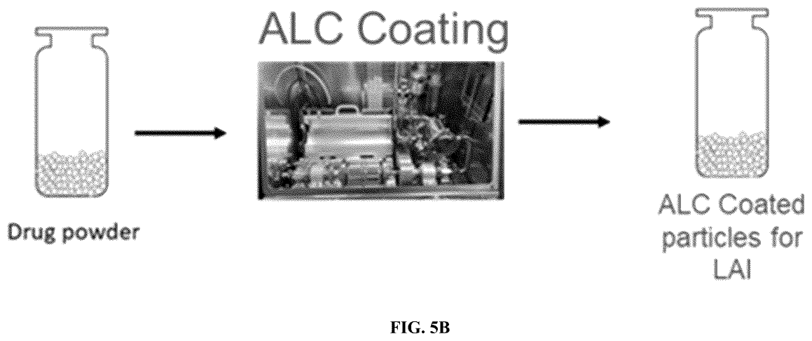

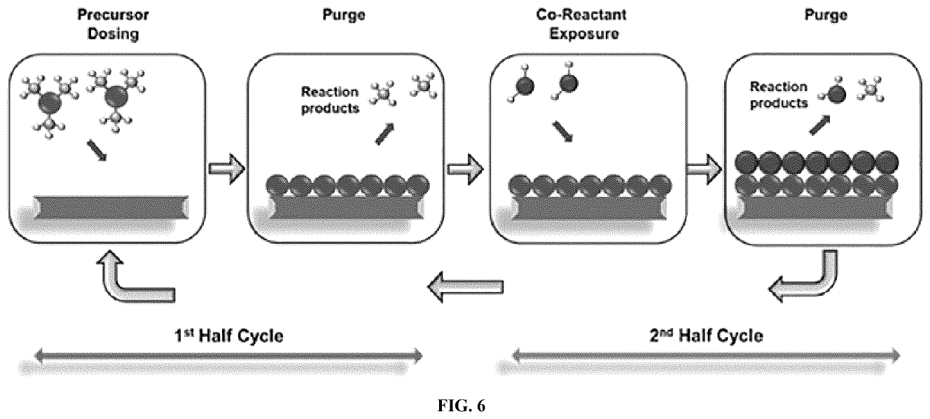

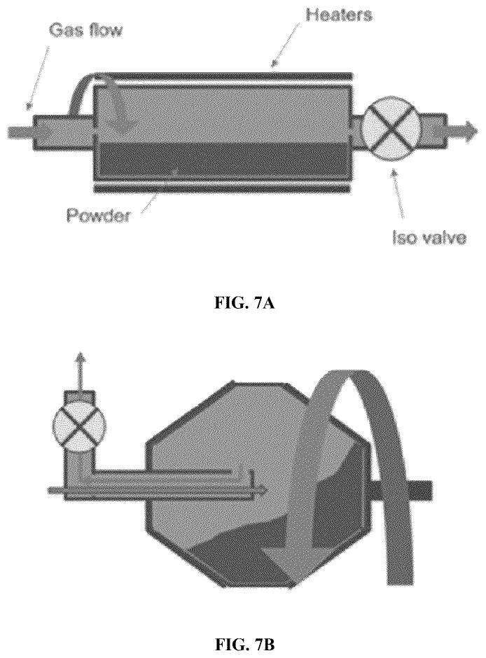

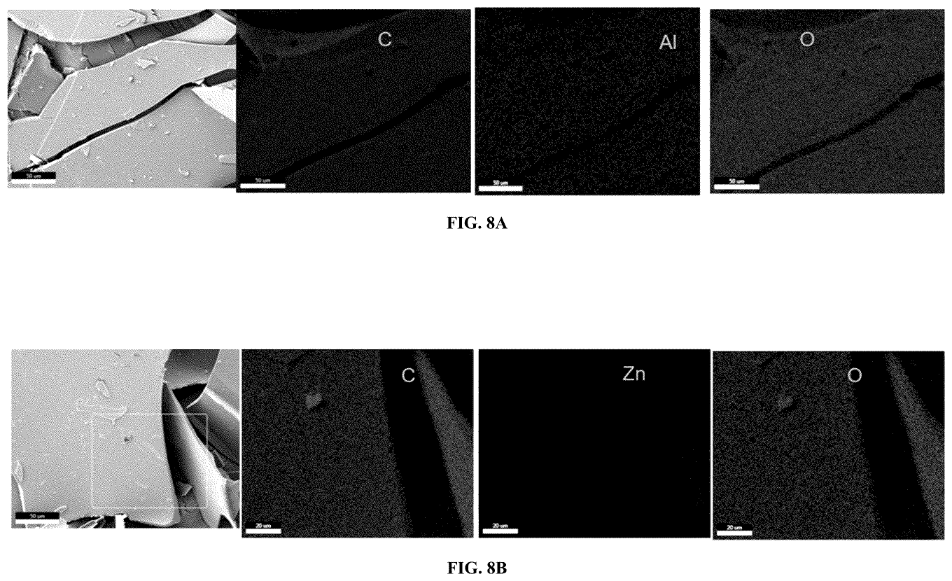

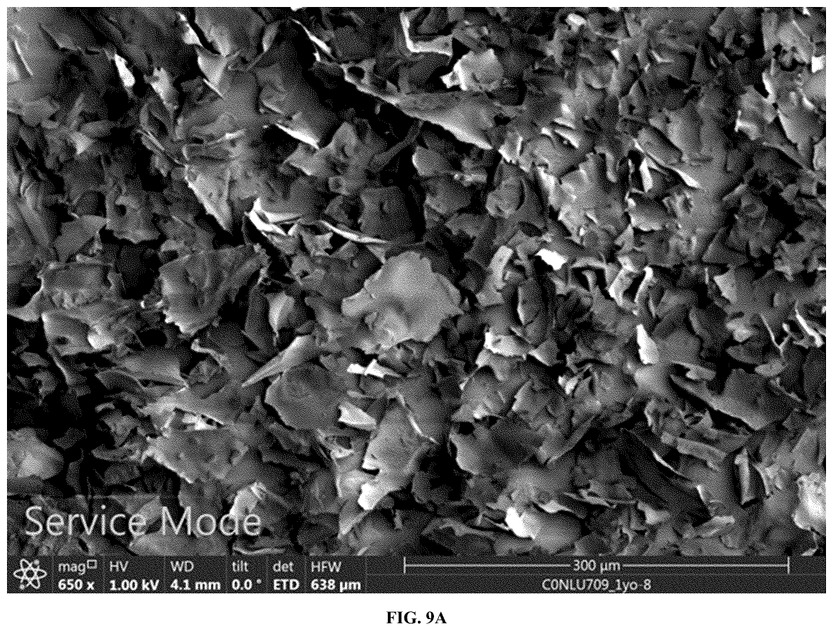

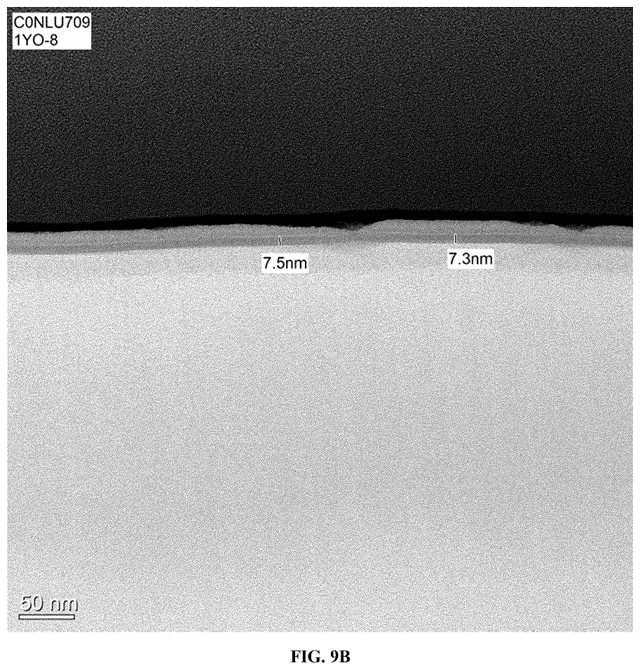

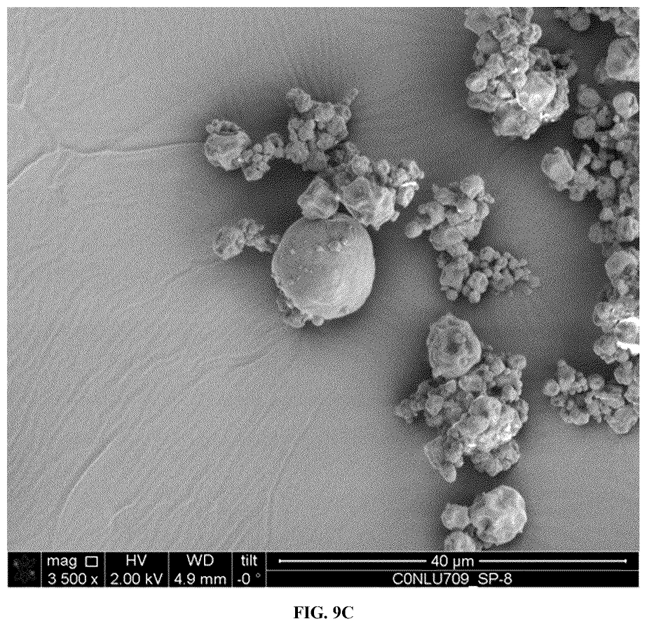

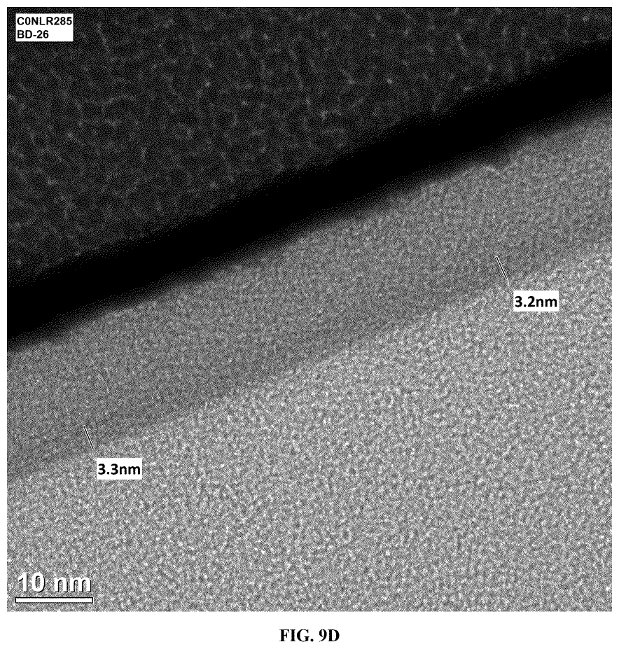

The term “reactor system” in its broadest sense includes all systems that could be used to perform vapor phase deposition or atomic layer deposition. An exemplary reactor system is illustrated in FIG. 1 and further described below.

The reactor system 10 can perform vapor phase deposition or atomic layer deposition. The reactor system 10 permits the process to be performed at higher (above 50° C., e.g., 50-100° C. or higher) or lower process temperature, e.g., below 50° C., e.g., at or below 25° C. For example, the reactor system 10 can form thin-film inorganic oxides on the particles primarily at temperatures of 40-80° C., e.g., 40° C. or 80° C. In general, the particles can remain or be maintained at such temperatures. This can be achieved by having the reactants and/or the interior surfaces of the reactor chamber (e.g., the chamber 20 and drum 40 discussed below) remain or be maintained at such temperatures.

Again, illustrating a vapor phase deposition or atomic layer deposition process, the reactor system 10 includes a stationary vacuum chamber 20 which is coupled to a vacuum pump 24 by vacuum tubing 22. The vacuum pump 24 can be an industrial vacuum pump sufficient to establish pressures less than 1 Torr, e.g., 1 to 100 mTorr, e.g., 50 mTorr. The vacuum pump 24 permits the chamber 20 to be maintained at a desired pressure and permits removal of reaction byproducts and unreacted process gases.

In operation, the reactor 10 performs the vapor phase deposition or atomic layer deposition process by introducing a gaseous oxidant and aluminum (or zinc) precursor into the chamber 20. The gaseous oxidant and aluminum (or zinc) precursor are introduced alternatively into the reactor. In addition, the reaction can be performed at low temperature conditions, such as below 80° C., e.g., below 50° C., below 30° C., or below 25° C. The operating temperature may be 50° C. The operating temperature may be above 5° C., above 10° C., above 15° C., above 20° C., above 25° C., above 30° C., above 35° C., above 40° C., above 45° C., above 50° C., above 56° C., above 60° C., above 65° C., above 70° C., above 75° C., or above 80° C. The operating temperature may be below 20° C., below 25° C., below 30° C., below 35° C., below 40° C., below 45° C., below 50° C., below 56° C., below 60° C., below 65° C., below 70° C., below 75° C., or below 80° C.

The chamber 20 is also coupled to a chemical delivery system 30. The chemical delivery system 30 includes three or more gas sources 32a, 32b, 32c coupled by respective delivery lines 34a, 34b, 34c and controllable valves 36a, 36b, 36c to the vacuum chamber 20. The chemical delivery system 30 can include a combination of restrictors, gas flow controllers, pressure transducers, and ultrasonic flow meters to provide controllable flow rate of the various gasses into the chamber 20. The chemical delivery system 30 can also include one or more temperature control components, e.g., a heat exchanger, resistive heater, heat lamp, etc., to heat or cool the various gasses before they flow into the chamber 20. Although FIG. 1 illustrates separate gas lines extending in parallel to the chamber for each gas source, two or more of the gas lines could be joined, e.g., by one or more three-way valves, before the combined line reaches the chamber 20.

One of the gas sources can provide an oxidant. In particular, a gas source can provide a vaporous or gaseous oxidant. For example, the oxidant can be ozone. As another example, the oxidant can be water vapor.

One of the gas sources can be an aluminum (or zinc) precursor. In particular, a gas source can provide a vaporous or gaseous aluminum (or zinc) precursor. For example, the aluminum precursor can be TMA.

One of the gas sources can provide a purge gas. In particular, the third gas source can provide a gas that is chemically inert to the oxidant and aluminum (or zinc) precursor, the coating, and the particles being processed. For example, the purge gas can be N₂, or a noble gas, such as argon.

A rotatable coating drum 40 is held inside the chamber 20. The drum 40 can be connected by a drive shaft 42 that extends through a sealed port in a side wall of the chamber 20 to a motor 44. The motor 44 can rotate the drum at speeds of 1 to 100 rpm. Alternatively, the drum can be directly connected to a vacuum source through a rotary union.

The particles to be coated, shown as a particle bed 50, are placed in an interior volume 46 of the drum 40. The drum 40 and chamber 20 can include sealable ports (not illustrated) to permit the particles to be placed into and removed from the drum 40.

The body of the drum 40 is provided by one or more of a porous material, a solid metal, and a perforated metal. The pores through the cylindrical side walls of the drum 40 can have a dimension of 1-10 μm.

In operation, one of the gasses flows into chamber 20 from the chemical delivery system 30 as the drum 40 rotates. A combination of pores (1-100 μm), holes (0.1-10 mm), or large openings in the coating drum 40 serve to confine the particles in the coating drum 40 while allowing rapid delivery of precursor chemistry and the pumping of byproducts or unreacted species. Due to the pores in the drum 40, the gas can flow between the exterior of the drum 40, i.e., the reactor chamber 20, and the interior of the drum 40. In addition, rotation of the drum 40 agitates the particles to expose new surfaces of the powder bed, ensuring a large surface area of the particles remains exposed to the process gas. This permits fast, uniform interaction of the particle surface with the process gas.

In some implementations, one or more temperature control components are integrated into the drum 40 to permit control of the temperature of the drum 40. For example, a resistive heater, a thermoelectric cooler, or other component can be in or on the side walls of the drum 40.

The reactor system 10 also includes a controller 60 coupled to the various controllable components, e.g., vacuum pump 24, gas distribution system 30, motor 44, a temperature control system, etc., to control operation of the reactor system 10. The controller 60 can also be coupled to various sensors, e.g., pressure sensors, flow meters, etc., to provide closed loop control of the pressure of the gasses in the chamber 20.

In general, the controller 60 can operate the reactor system 10 in accord with a “recipe.” The recipe specifies an operating value for each controllable element as a function of time. For example, the recipe can specify the times during which the vacuum pump 24 is to operate, the times of and flow rate for each gas source 32a, 32b, 32c, the rotation rate of the motor 44, etc. The controller 60 can receive the recipe as computer-readable data (e.g., that is stored on a non-transitory computer readable medium).

The controller 60 and other computing device parts of systems described herein can be implemented in digital electronic circuitry, or in computer software, firmware, or hardware. For example, the controller can include a processor to execute a computer program as stored in a computer program product, e.g., in a non-transitory machine-readable storage medium. Such a computer program (also known as a program, software, software application, or code) can be written in any form of programming language, including compiled or interpreted languages, and it can be deployed in any form, including as a standalone program or as a module, component, subroutine, or other unit suitable for use in a computing environment. In some implementations, the controller 60 is a general-purpose programmable computer. In some implementations, the controller can be implemented using special purpose logic circuitry, e.g., an FPGA (field programmable gate array) or an ASIC (application specific integrated circuit).

### Operation

Initially, uncoated particles (e.g., polypeptide particles) are loaded into the drum 40 in the reactor system 10. Once any access ports are sealed, the controller 60 operates the reactor system 10 according to the recipe in order to form the thin-film inorganic oxides on the particles.

The uncoated particles may be coated with an inorganic oxide coating. The uncoated particles may be coated with two reactants. For example, the uncoated particles may be coated with a first reactant in the form of an inorganic precursor and a second reactant in the form of an oxidant. The inorganic oxide may be aluminum oxide, zinc oxide, titanium oxide, or silicon oxide. The inorganic oxide may be aluminum oxide. The inorganic precursor may be trimethylaluminium (TMA). The inorganic oxide may be zinc oxide. The inorganic precursor may be diethylzinc. The inorganic oxide may be titanium oxide. The inorganic precursor may be titanium tetrachloride (TiCl₄). The inorganic oxide may be silicon oxide. The inorganic precursor may be 1,2-Bis(diisopropylamino) disilane (BDIPADS) or silicon tetrachloride. The oxidant may be water or ozone.

In particular, the two reactant gases are alternately supplied to the chamber 20, with each step of supplying a reactant gas followed by a purge cycle in which the inert gas is supplied to the chamber 20 to force out the reactant gas and by-products used in the prior step. Moreover, one or more of the gases (e.g., the reactant gases and/or the inert gas) can be supplied in pulses in which the chamber 20 is filled with the gas to a specified pressure, a delay time is permitted to pass, and the chamber is evacuated by the vacuum pump 24 before the next pulse commences. In particular, the controller 60 can operate the reactor system 10 as follows. In a first reactant cycle (called a half-cycle), while the motor 44 rotates the drum 40 to agitate the particles 50:
i) The gas distribution system 30 is operated to flow the first reactant gas, e.g., TMA, from the source 32a into the chamber 20 until a first specified pressure is achieved. The specified pressure can be 0.1 Torr to half of the saturation pressure of the reactant gas.
ii) Flow of the first reactant is halted, and a specified delay time is permitted to pass, e.g., as measured by a timer in the controller. This permits the first reactant to flow through the particle bed in the drum 40 and react with the surface of the particles 50 inside the drum 40.
iii) The vacuum pump 50 evacuates the chamber 20, e.g., down to pressures below 1 Torr, e.g., to 1 to 100 mTorr, e.g., 50 mTorr.

These steps (i)-(iii) can be repeated a number of times set by the recipe, e.g., two to ten times, e.g., six times.

Next, in a first purge cycle, while the motor 44 rotates the drum to agitate the particles 50:
iv) The gas distribution system 30 is operated to flow the inert gas, e.g., N₂, from the source 32c into the chamber 20 until a second specified pressure is achieved. The second specified pressure can be 1 to 100 Torr.
v) Flow of the inert gas is halted, and a specified delay time is permitted to pass, e.g., as measured by the timer in the controller. This permits the inert gas to flow through the pores in the drum 40 and diffuse through the particles 50 to displace the reactant gas and any vaporous by-products.
vi) The vacuum pump 50 evacuates the chamber 20, e.g., down to pressures below 1 Torr, e.g., to 1 to 500 mTorr, e.g., 50 mTorr. These steps (iv)-(vi) can be repeated a number of times set by the recipe, e.g., six to twenty times, e.g., sixteen times. Taken together steps (iv)-(vi) are called a pump-purge cycle.

In a second reactant half-cycle, while the motor 44 rotates the drum 40 to agitate the particles 50:

vii) The gas distribution system 30 is operated to flow the second reactant gas, e.g., H₂O, from the source 32b into the chamber 20 until a third specified pressure is achieved. The third pressure can be 0.1 Torr to half of the saturation pressure of the reactant gas.
viii) Flow of the second reactant is halted, and a specified delay time is permitted to pass, e.g., as measured by the timer in the controller. This permits the second reactant to flow through the pores in the drum 40 and react with the surface of the particles 50 inside the drum 40.
ix) The vacuum pump 50 evacuates the chamber 20, e.g., down to pressures below 1 Torr, e.g., to 1 to 500 mTorr, e.g., 50 mTorr.

These steps (vii)-(ix) can be repeated a number of times set by the recipe, e.g., two to ten times, e.g., six times.

Next, a second purge cycle is performed. This second purge cycle can be identical to the first purge cycle, or can have a different number of repetitions of the steps (iv)-(vi) and/or different delay time and/or different pressure.

The cycle of the first reactant half-cycle, first purge cycle, second reactant half cycle and second purge cycle can be repeated a number of times set by the recipe, e.g., one to ten times.

As noted above, the coating process can be performed at a low process temperature, e.g., below 80° C., e.g., at or below 50° C., at or below 35° C., or at or below 25° C. The operating temperature may be 50° C. The operating temperature may be above 5° C., above 10° C., above 15° C., above 20° C., above 25° C., above 30° C., above 35° C., above 40° C., above 45° C., above 50° C., above 56° C., above 60° C., above 65° C., above 70° C., above 75° C., or above 80° C. (e.g. 20° C. to 80° C.). The operating temperature may be below 20° C., below 25° C., below 30° C., below 35° C., below 40° C., below 45° C., below 50° C., below 56° C., below 60° C., below 65° C., below 70° C., below 75° C., or below 80° C. In particular, the particles can remain or be maintained at such temperatures during all of steps (i)-(ix) noted above. In general, the temperature of the interior of the reactor chamber does not exceed 80° C. during of steps (i)-(ix). This can be achieved by having the oxidant gas, precursor gas and inert gas be injected into the chamber at such temperatures during the respective cycles. In addition, physical components of the chamber can remain or be maintained at such temperatures, e.g., using a cooling system, e.g., a thermoelectric cooler, if necessary.

The uncoated particle may be coated with one or more inorganic oxide coatings. The uncoated particle may be coated with a combination of aluminum oxide, titanium oxide, zinc oxide and/or silicon oxide coatings. For example, the uncoated particles may be first coated with an aluminum oxide layer and then coated with a silicon oxide layer. The uncoated particle may be first coated with an aluminum oxide layer, then coated with a silicon oxide layer, and then coated with an aluminum oxide layer. The uncoated particle may be coated with supercycles, each supercycle comprising a few cycles (e.g., 1-10) of a first metal oxide coating (e.g., aluminum oxide) and a few cycles of a second metal oxide (e.g., zinc oxide).

Exemplary methods for applying aluminum oxide, titanium oxide, zinc oxide, silicon oxide coatings, multiple coating layers and supercycle coating methods are provided below.

### Methods for Aluminum Oxide Coating

In one aspect, the disclosure provides methods for preparing coated particles comprising a polypeptide particle (a polypeptide-containing core) encapsulated by aluminum oxide coating.

The aluminum oxide coating can be performed on a rotary powder coating chamber. The operating temperature can be 35-50° C. or about 50° C. For each aluminum oxide coating process, one precursor may be introduced to reach a pressure of 0.3-2 torr for a hold time of 60 seconds, before nitrogen gas is used to purge the excess reactants and side products. The second precursor may then be introduced to reach a pressure of 2-8 torr for a hold time of 60 seconds, before nitrogen gas is used to purge the excess reactants and side products. This completes one coating cycle. Desired cycle numbers can be decided and aluminum oxide coating can be coated in repeated cycles to obtain a desired thickness.

The aluminum oxide coating can be applied using vapor phase deposition as described herein. The aluminum precursors can be trimethylaluminum (TMA). The oxidant can be water.

A first exemplary aluminum oxide coating method includes the sequential steps of: (a) loading the uncoated particles into a reactor, (b) applying a vaporous or gaseous aluminum precursor (e.g., TMA) to the substrate in the reactor, (c) performing one or more pump-purge cycles of the reactor using inert gas, (d) applying a vaporous or gaseous oxidant (e.g., water) to the substrate in the reactor, and (c) performing one or more pump-purge cycles of the reactor using inert gas. The sequential steps (b)-(c) may be repeated one or more times to increase the total thickness of the aluminum oxide that enclose the solid core of the coated particles. The reactor pressure may be allowed to stabilize following step (a), step (b), and/or step (d). The reactor contents may be agitated prior to and/or during step (b), step (c), and/or step (c). A subset of vapor or gaseous content may be pumped out prior to step (c) and/or step (c).

A second exemplary aluminum oxide coating method includes (e.g., consists of) the sequential steps of (a) loading the uncoated particles into a reactor, (b) reducing the reactor pressure to less than 50m Torr, (c) agitating the reactor contents until the reactor contents have a desired moisture content, (d) pressurizing the reactor to at least 0.3 Torr by adding a vaporous or gaseous aluminum precursor (e.g., TMA), (c) allowing the reactor pressure to stabilize, (f) agitating the reactor contents, (g) pumping out a subset of vapor or gaseous content and determining when to stop pumping based on analysis of content in reactor, (h) performing a sequence of pump-purge cycles of the reactor using insert gas, (i) pressuring the reactor to 2 Torr by adding a vaporous or gaseous oxidant (e.g., water), (j) allowing the reactor pressure to stabilize, (k) agitating the reactor contents, (l) pumping out a subset of vapor or gaseous content and determining when to stop pumping based on analysis of content in reactor and (m) performing a sequence of pump-purge cycles of the reactor using insert gas. The sequential steps (b)-(m) may be repeated one or more times to increase the total thickness of the one or more aluminum oxide materials that enclose the solid core of the coated particles.

### Methods for Titanium Oxide Coating

In one aspect, the disclosure provides methods for preparing coated particles comprising a polypeptide particle (a polypeptide-containing core) encapsulated by titanium oxide coating.

The titanium oxide coating can be performed on a rotary powder coating chamber. The operating temperature can be 35-50° C. or about 50° C. For each titanium oxide coating process, one precursor may be introduced to reach a pressure of 0.3-2 torr for a hold time of 60 seconds, before nitrogen gas is used to purge the excess reactants and side products. The second precursor may then be introduced to reach a pressure of 2-8 torr for a hold time of 60 seconds, before nitrogen gas is used to purge the excess reactants and side products. This completes one coating cycle. Desired cycle numbers can be decided and titanium oxide coating can be coated in repeated cycles to obtain a desired thickness.

The titanium oxide coating can be applied using vapor phase deposition as described herein. The titanium precursor can be titanium tetrachloride (TiCl₄), tetrakis (dimethylamino) titanium (TDMAT), tetrakis (diethylamino) titanium (TDEAT), or tetrakis (ethylmethylamino) titanium (TEMAT). The oxidant can be water or ozone. The titanium precursor may be titanium tetrachloride and the oxidant may be water.

A first exemplary titanium oxide coating method includes the sequential steps of: (a) loading the uncoated particles into a reactor, (b) applying a vaporous or gaseous titanium precursor (e.g., TiCl₄) to the substrate in the reactor, (c) performing one or more pump-purge cycles of the reactor using inert gas, (d) applying a vaporous or gaseous oxidant (e.g., water) to the substrate in the reactor, and (e) performing one or more pump-purge cycles of the reactor using inert gas. The sequential steps (b)-(c) may be repeated one or more times to increase the total thickness of the titanium oxide that enclose the solid core of the coated particles. The reactor pressure may be allowed to stabilize following step (a), step (b), and/or step (d). The reactor contents may be agitated prior to and/or during step (b), step (c), and/or step (c). A subset of vapor or gaseous content may be pumped out prior to step (c) and/or step (e).

A second exemplary titanium oxide coating method includes (e.g., consists of) the sequential steps of (a) loading the uncoated particles into a reactor, (b) reducing the reactor pressure to less than 50 m Torr, (c) agitating the reactor contents until the reactor contents have a desired moisture content, (d) pressurizing the reactor to at least 0.3 Torr by adding a vaporous or gaseous titanium precursor (e.g., TiCl₄), (c) allowing the reactor pressure to stabilize, (f) agitating the reactor contents, (g) pumping out a subset of vapor or gaseous content and determining when to stop pumping based on analysis of content in reactor, (h) performing a sequence of pump-purge cycles of the reactor using insert gas, (i) pressuring the reactor to 2 Torr by adding a vaporous or gaseous oxidant (e.g., water), (j) allowing the reactor pressure to stabilize, (k) agitating the reactor contents, (l) pumping out a subset of vapor or gaseous content and determining when to stop pumping based on analysis of content in reactor and (m) performing a sequence of pump-purge cycles of the reactor using insert gas. The sequential steps (b)-(m) may be repeated one or more times to increase the total thickness of the one or more titanium oxide materials that enclose the solid core of the coated particles.

### Methods for Zinc Oxide Coating

In one aspect, the disclosure provides methods for preparing coated particles comprising a polypeptide particle (a polypeptide-containing core) encapsulated by zinc oxide coating.

The first exemplary method includes the sequential steps of: (a) loading the uncoated particles into a reactor, (b) applying a vaporous or gaseous zinc precursor to the substrate in the reactor, (c) performing one or more pump-purge cycles of the reactor using inert gas, (d) applying a vaporous or gaseous oxidant (e.g., water) to the substrate in the reactor, and (c) performing one or more pump-purge cycles of the reactor using inert gas. The sequential steps (b)-(c) may be repeated one or more times to increase the total thickness of the zinc oxide that enclose the solid core of the coated particles. The reactor pressure may be allowed to stabilize following step (a), step (b), and/or step (d). The reactor contents may be agitated prior to and/or during step (b), step (c), and/or step (e). A subset of vapor or gaseous content may be pumped out prior to step (c) and/or step (e).

The second exemplary method includes (e.g., consists of) the sequential steps of (a) loading the uncoated particles into a reactor, (b) reducing the reactor pressure to less than 50 m Torr, (c) agitating the reactor contents until the reactor contents have a desired moisture content, (d) pressurizing the reactor to at least 0.3 Torr by adding a vaporous or gaseous zinc precursor, (e) allowing the reactor pressure to stabilize, (f) agitating the reactor contents, (g) pumping out a subset of vapor or gaseous content and determining when to stop pumping based on analysis of content in reactor, (h) performing a sequence of pump-purge cycles of the reactor using insert gas, (i) pressuring the reactor to 2 Torr by adding a vaporous or gaseous oxidant (e.g., water), (j) allowing the reactor pressure to stabilize, (k) agitating the reactor contents, (l) pumping out a subset of vapor or gaseous content and determining when to stop pumping based on analysis of content in reactor and (m) performing a sequence of pump-purge cycles of the reactor using insert gas. The sequential steps (b)-(m) may be repeated one or more times to increase the total thickness of the one or more zinc oxide materials that enclose the solid core of the coated particles.

The step of performing atomic layer coating may comprise: (b1) loading the uncoated particles into a reactor; (b2) applying a vaporous or gaseous zinc precursor to the particles in the reactor; (b3) performing one or more pump-purge cycles of the reactor using inert gas; (b4) applying a vaporous or gaseous oxidant (e.g., water) to the particles in the reactor; and (b5) performing one or more pump-purge cycles of the reactor using inert gas. Steps (b2)-(b5) may be performed two or more times to increase the total thickness of the zinc oxide layer before step (c) is performed.

The reactor pressure may be allowed to stabilize following step (b1), step (b2), and/or step (b4). The reactor contents may be agitated prior to and/or during step (b1), step (b3), and/or step (b5). A subset of vapor or gaseous content may be pumped out prior to step (b3) and/or step (b5). Step (b) may take place at a temperature between 45° C. and 55° C.

### Methods for Silicon Oxide Coating

In one aspect, the disclosure provides methods for preparing coated particles comprising a polypeptide particle (a polypeptide-containing core) encapsulated by silicon oxide coating.

The silicon oxide coating can be applied using vapor phase deposition as described herein. The silicon precursors can be silicon tetrachloride or 1,2-Bis (diisopropylamino) disilane (BDIPADS). The oxidant can be water or ozone.

A first exemplary silicon oxide coating method includes the sequential steps of: (a) loading the uncoated particles into a reactor, (b) applying a vaporous or gaseous silicon precursor (e.g., silicon tetrachloride, BDIPADS) to the substrate in the reactor, (c) performing one or more pump-purge cycles of the reactor using inert gas, (d) applying a vaporous or gaseous oxidant (e.g., ozone) to the substrate in the reactor, and (c) performing one or more pump-purge cycles of the reactor using inert gas. The sequential steps (b)-(c) may be repeated one or more times to increase the total thickness of the silicon oxide that enclose the solid core of the coated particles. The reactor pressure may be allowed to stabilize following step (a), step (b), and/or step (d). The reactor contents may be agitated prior to and/or during step (b), step (c), and/or step (c). A subset of vapor or gaseous content may be pumped out prior to step (c) and/or step (c).

A second exemplary silicon oxide coating method includes (e.g., consists of) the sequential steps of (a) loading the uncoated particles into a reactor, (b) reducing the reactor pressure to less than 50 m Torr, (c) agitating the reactor contents until the reactor contents have a desired moisture content, (d) pressurizing the reactor to at least 0.3 Torr by adding a vaporous or gaseous silicon precursor (e.g., silicon tetrachloride, BDIPADS), (c) allowing the reactor pressure to stabilize, (f) agitating the reactor contents, (g) pumping out a subset of vapor or gaseous content and determining when to stop pumping based on analysis of content in reactor, (h) performing a sequence of pump-purge cycles of the reactor using insert gas, (i) pressuring the reactor to 2 Torr by adding a vaporous or gaseous oxidant (e.g., ozone), (j) allowing the reactor pressure to stabilize, (k) agitating the reactor contents, (l) pumping out a subset of vapor or gaseous content and determining when to stop pumping based on analysis of content in reactor and (m) performing a sequence of pump-purge cycles of the reactor using insert gas. The sequential steps (b)-(m) may be repeated one or more times to increase the total thickness of the one or more silicon oxide materials that enclose the solid core of the coated particles.

Methods for Preparing a Coated Particle with Multiple Coating Layers

In some cases, the coating is applied using two or more different processes, for example, a silicon oxide coating process and an aluminum oxide coating process. The two or more different processes can be combined, e.g., to provide a distinct, inner aluminum oxide coating layer adjacent to the polypeptide particle and a distinct, outer silicon oxide coating layer that encloses the particle, including the inner layer. In some cases, another coating layer can be applied surrounding the outer coating layer. Thus, there can be a distinct inner layer of a first metal or metalloid oxide, a distinct intermediate layer of a second metal or metalloid oxide and a third outer layer of the first (or a third) metalloid oxide. In general, the formation of such distinct coating layers entails at least 10 cycles of vapor phase deposition for each distinct coating layer, generating a coating layer that is about 2.5-100 nanometers thick.

### Methods for Preparing a Coated Particles by a Supercycle Process

In contrast to other methods for applying an organic oxide coating using vapor phase deposition, the supercycle process entails carrying out a small number (e.g., 1, 2, 3, 4, 5, 6, 7, 8, 9 or 10) of cycles with a first precursor-oxidant pair, e.g., trimethylaluminum and water, and then carrying out a small number of cycles (e.g., 1, 2, 3, 4, 5, 6, 7, 8, 9 or 10) with a different precursor-oxidant pair (e.g., diethyl zinc and water) to create a layer containing three elements (e.g., aluminum, zinc and oxygen). The sequence of, for example, one cycle with a first precursor-oxidant pair and 3 cycles with second, different precursor-oxidant pair is called a “supercycle” and can be repeated a number of times, for example, 2-10 times, 5-20 times or 2-50 or more times. The entire coating can be composed of such a layer. However, the process can continue by using vapor phase deposition to apply a conventional, two element inorganic oxide layer, e.g., a zinc oxide layer. Instead, the process can continue with a second set of supercycles each of which can have a different number of cycles using the first precursor-oxidant pair and the second precursor-oxidant pairs (e.g., 2 cycles with the first precursor-oxidant pair and 10 cycles with second precursor-oxidant pair). Carried out in this specific manner, the process created two supercycle layers adjacent to each other that both contain the same three elements (e.g., aluminum, zinc and oxygen), but in differing proportions. In another variation, the process can continue with a second set of supercycles using different precursor-oxidant pairs. When two different precursor-oxidant pairs are used, the oxidants can be the same or different.

When carried out with two different precursor-oxidant pairs (e.g., trimethylaluminum/water and diethyl zinc/water) that differ in the metal or metalloid, the supercycle process creates a relatively uniform layer primarily composed of three elements (e.g., aluminum, zinc and oxygen (“AZO”)). This can be considered essentially a ternary oxide. When the supercycle process is carried out to create a layer, the layer can include small regions composed of two elements (e.g., aluminum and oxygen or zinc and oxygen). For example, when more zinc oxide precursor is used, there may be a small region composed of zinc oxide.

However, overall, the layer would still be composed of three elements. The method permits fine tuning of the characteristics of the coating. This is, at least in part, due to the ability to combine aspects of two different inorganic oxides such as aluminum oxide and zinc oxide. For example, an aluminum oxide layer is generally relatively dense and amorphous and can provide a barrier that is relatively less permeable to, for example, water. In contrast, a zinc oxide layer is generally relatively crystalline and has grain boundaries that make the coating relatively porous compared to, for example, an aluminum oxide coating. Combining these properties has the advantage of creating a layer that is relatively less permeable to water but has a relatively small amount of aluminum, which can be desirable in certain circumstances.

As another example, the supercycle process can be used to create a relatively uniform layer that primarily composed of aluminum, silicon and oxygen (“ASO”) and, sometimes, regions compound of aluminum and oxygen or silicon and oxygen. For example, when more silicon oxide precursor is used, there may be a small region composed of silicon oxide. However, overall the layer would still be composed of three elements. An ASO layer can be created by carrying out just a few (e.g., 1, 2, 3, 4, 5, 6, 7, 8, 9 or 10) cycles with a first precursor-oxidant pair, e.g., trimethylaluminum and water, and then carrying out a number of cycles (e.g., 1, 2, 3, 4, 5, 6, 7, 8, 9 or 10) with a different precursor-oxidant pair (e.g., SiCl₄ and water). The process can then continue with a second set of supercycles each of which can have a different number of cycles using the first precursor-oxidant pair and the second precursor-oxidant pairs (e.g., 2 cycles with the first precursor-oxidant pair and 10 cycles with second precursor oxidant pair). When carried out with two different precursor-oxidant pairs (e.g., trimethylaluminum/water and SiCl/water), this process creates a coating that has a relatively high proportion of ternary compound composed of three elements (e.g., aluminum, silicon and oxygen (ASO)) and a relatively small proportion of binary compound composed of two elements (e.g., aluminum and oxygen or silicon and oxygen). The first precursor-oxidant pair can also be trimethylaluminum and ozone. The second precursor-oxidant pair can also be 1,2-Bis (diisopropylamino) disilane (BDIPADS) and ozone. The second precursor-oxidant pair can also be Diisopropylaminosilane (DIPAS) and ozone.

This process described herein is referred herein as a “supercycle vapor phase deposition process” or simply a “supercycle” process. When such a process is carried out with a combination of aluminum oxide precursor and zinc oxide precursor, the resulting layer coating is referred to as an aluminum/zinc oxide (AZO) layer. When such a process is carried out with a combination of aluminum oxide precursor and a silicon oxide precursor, the resulting layer is referred to as an aluminum/silicon oxide (ASO) layer. When such a process is carried out with a combination of zinc oxide precursor and a silicon oxide precursor, the resulting layer is referred to as an aluminum/silicon oxide (ZSO) layer. A layer created only with an aluminum oxide precursor and an oxident is called an aluminum oxide layer or “AlOx” layer. A layer created only with a zinc oxide precursor and an oxidant is called a zinc oxide layer or “ZnOx” coating. A layer created only with a silicon oxide precursor and an oxidant is called a silicon oxide coating or “SiOx” layer.

Of course, a supercycle coating process can be combined with a more conventional coating process. Thus, a particle can have an inner layer produced using a supercycle process and an outer layer that is a distinct layer (i.e., not a ternary compound layer). The two types of coating layers can be applied in reverse order to produce a particle having an inner layer that is a distinct layer (i.e., not a ternary compound layer) and outer layer that is a ternary compound layer produced using a supercycle process.

In one example, the disclosure provides methods to prepare a coated particle that has a polypeptide containing core and at least one coating layer applied using supercycles, each supercycle having a first number of first cycles and a second number of second cycles. The methods include the sequential steps of: (a) providing uncoated particles; (b) performing a first number of first cycles; using first inorganic oxide precursor and (c) performing a second number of second cycles using a second inorganic oxide precursor, wherein the first and second inorganic oxide precursors are for forming different inorganic oxides (e.g., the first precursor can be aluminum oxide precursor and the second precursor can be zinc oxide precursor). The vaporous or gaseous oxidant used in the first and second cycles can be the same or different.

The step of performing a first number of first cycles (step (b)) comprises: (b1) applying a vaporous or gaseous first inorganic oxide precursor to the particles in the reactor by pulsing the vaporous or gaseous first inorganic oxide precursor into the reactor; (b2) performing one or more pump-purge cycles of the reactor using an inert gas; (b3) applying a vaporous or gaseous oxidant to the particles in the reactor by pulsing the oxidant into the reactor; and (b4) performing one or more pump-purge cycles of the reactor using an inert gas.

The step of performing a second number of second cycles (step (c)) comprises: (c1) applying a vaporous or gaseous second inorganic oxide precursor to the particles in the reactor by pulsing the vaporous or gaseous second inorganic oxide precursor into the reactor; (c2) performing one or more pump-purge cycles of the reactor using an inert gas; (c3) applying a vaporous or gaseous oxidant to the particles in the reactor by pulsing the oxidant into the reactor; and (c4) performing one or more pump-purge cycles of the reactor using an inert gas.

The steps (b)-(c) constitutes a supercycle. Steps (b)-(c) can be performed two or more times to increase the total thickness of the coating. The particles can be agitated prior to and/or during step (a). The reactor pressure can be allowed to stabilize following step (b1), step (b2), step (b3) and/or step (b4). The reactor pressure can be allowed to stabilize following step (c1), step (c2), step (c3) and/or step (c4).

For example, aluminum oxide and zinc oxide precursors can be applied using supercycles to create an AZO layer The aluminum precursor can be trimethylaluminum (TMA). The zinc precursor can be diethylzinc (DEZ) or zinc tetrachloride. The silicon precursor can be SiCl₄, Tris (tertpentoxy) silanol, diisopropylamino silane (DIPAS) or 1,2-Bis(diisopropylamino) disilane (BDIPADS).

### Coated Particles

In one aspect, the disclosure provides coated particles where a coating is applied to the uncoated particles (polypeptide-containing cores) through the vapor phase deposition method described herein. The coated particles may be coated polypeptide particles (particles containing one or more polypeptides). The coated particles may be coated antibody particles (particles of one or more antibodies). The coated particles may further contain one or more pharmaceutically acceptable excipients.

The coated particles may have a reduced surface charge, as compared to uncoated particles. The coated particles may have enhanced hydrophilicity, as compared to uncoated particles. The coated particles may be less prone to aggregation, as compared to uncoated particles. The coated particles may have an improved flowability compared to uncoated particles. Applying the coating may improve the wettability and/or dispersibility of the uncoated particles. Applying the coating may improve the dispersibility, but not the wettability of the uncoated particles. Applying the coating may slow the release of the active ingredient in the uncoated particles.

The structure of the polypeptide (e.g., antibody) can be assessed by X-Ray Diffraction (XRD) analysis. There are no significant changes in XRD signals before and after the coating process. There may be no significant structural change in the polypeptide (e.g., antibody) after the coating process.

The structure of polypeptide (e.g., antibody) can be assessed by Fourier-transform infrared (FTIR) analysis. There may be no significant changes in FTIR signals before and after the coating process.

The composition of the coated particles can be assessed by Thermogravimetric Analysis (TGA) analysis. The amount of inorganic component may constitute more than 0.1%, more than 0.2%, more than 0.3%, more than 0.4%, more than 0.5%, more than 0.6%, more than 0.7%, more than 0.8%, more than 0.9%, more than 1%, more than 1.2%, more than 1.4%, more than 1.6%, more than 1.8%, more than 2%, more than 2.2%, more than 2.4%, more than 2.6%, more than 2.8%, more than 3%, more than 3.2%, more than 3.4%, more than 3.6%, more than 3.8%, more than 4%, more than 4.2%, more than 4.4%, more than 4.6%, more than 4.8%, more than 5%, more than 6%, more than 7%, more than 8%, more than 9%, more than 10%, more than 12%, more than 14%, more than 16%, more than 18%, or more than 20% wt/wt of the coated particles.

Weight percent of inorganic oxides in the coated particles may be less than 0.1%, less than 0.2%, less than 0.3%, less than 0.4%, less than 0.5%, less than 0.6%, less than 0.7%, less than 0.8%, less than 0.9%, less than 1%, less than 1.2%, less than 1.4%, less than 1.6%, less than 1.8%, less than 2%, less than 2.2%, less than 2.4%, less than 2.6%, less than 2.8%, less than 3%, less than 3.2%, less than 3.4%, less than 3.6%, less than 3.8%, less than 4%, less than 4.2%, less than 4.4%, less than 4.6%, less than 4.8%, less than 5%, less than 6%, less than 7%, less than 8%, less than 9%, less than 10%, less than 12%, less than 14%, less than 16%, less than 18%, or less than 20% wt/wt of the coated particles. The weight percent of inorganic oxides in the coated particles may be 0.1%-20%, 0.5%-10%, 1%-10%, 1%-5%, 2%-5%, 1%-4%, 1%-3%, or 2%-4% wt/wt of the coated particles. The amount of inorganic component may constitute about 1%-20% wt/wt of the coated particles.

The morphology of the coated particles can be assessed by Transmission Electron Microscopy (TEM) analysis or Scanning Electron Microscopy (SEM) analysis. There may be no obvious change in particle size before and after the coating process. There may be no obvious morphology change before and after the coating process.

The coated particles may exhibit increased hydrophobicity comparing to the uncoated particles. The coated particles may exhibit increased powder flowability (“FFc”) comparing to the uncoated particles. The coated particles may exhibit increased bulk density comparing to the uncoated particles.

### EXAMPLES

The disclosure is further described in the following examples, which do not limit the scope of the disclosure described in the claims.

### Example 1

### The Preparation of a High Concentration Antibody Formulation (HCAF)

An exemplary HCAF is prepared by coating antibody particles with aluminum oxide. First, the antibody particles are created by lyophilization. Second, the antibody particles are coated by an aluminum oxide coating, by the sequential steps of:
(a) Loading particles comprising the antibody into a rotatory reactor;
(b) Pulsing an aluminum precursor (e.g., TMA) with a holding time of 1 minute;
(c) Purging the reactor with an inert gas to remove the aluminum precursor;
(d) Pulsing an oxidant (e.g., water) into the reactor, with a holding time of 1 minute;
(e) Purging the reactor with an inert gas to remove extra oxidant (e.g., water).

In some cases, the steps of (b)-(c) were repeated more than once to increase the total thickness of the aluminum oxide that enclose the antibody particle.

Finally, the coated particles are mixed with a pharmaceutically acceptable excipient to create the high concentration antibody formulation.

### Example 2

### The Preparation of a High Concentration Antibody Formulation (HCAF)

An exemplary HCAF is prepared by coating antibody particles with zinc oxide. First, the antibody particles are created by lyophilization. Second, the antibody particles are coated by the below coating method.

The method for creating a zinc oxide coating comprised the sequential steps of:
(f) Loading particles comprising the antibody into a rotatory reactor;
(g) Pulsing a zinc precursor (e.g., DEZ) with a holding time of 1 minute;
(h) Purging the reactor with an inert gas to remove the zinc precursor;
(i) Pulsing an oxidant (e.g., water) into the reactor, with a holding time of 1 minute;
(j) Purging the reactor with an inert gas to remove extra oxidant (e.g., water).

In some cases, the steps of (b)-(e) were repeated more than once to increase the total thickness of the zinc oxide that enclose the antibody particle.

Finally, the coated particles are mixed with a pharmaceutically acceptable excipient to create the high concentration antibody formulation.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A polypeptide formulation, comprising:
(a) a coated particle comprising a core comprising a polypeptide, and a coating layer comprising an inorganic oxide layer enclosing the core; and
(b) water,
wherein the inorganic oxide layer comprises an inorganic oxide that comprises aluminum, zinc, silicon, and/or titanium, wherein the polypeptide formulation comprises more than 100 mg/ml polypeptide.

2. The polypeptide formulation of claim 1, wherein the polypeptide formulation comprises more than 150 mg/ml polypeptide.

3. The polypeptide formulation of claim 1, wherein the polypeptide formulation comprises more than 100 mg/ml coated particles.

4. The polypeptide formulation of claim 1, wherein the coating layer is 0.1 nm-120 nm thick.

5. The polypeptide formulation of claim 1, wherein the coating layer is 5 nm-15 nm thick.

6. The polypeptide formulation of claim 1, wherein the coating layer is conformal and pin-hole free.

7. The polypeptide formulation of claim 1, wherein the coated particle comprises 1-20% wt/wt inorganic oxide.

8. (canceled)

9. The polypeptide formulation of claim 1, wherein the coated particle consists of the core and the coating layer.

10. The polypeptide formulation of claim 1, wherein the coated particles have a D50 on a volume average basis of 100 nm-30 micrometers.

11. The polypeptide formulation of claim 1, wherein the coated particles have a D50 on a volume average basis of 1-25 μm.

12. The polypeptide formulation of claim 1, wherein the coated particles have a median particle size, on a volume average basis, between 0.1 μm and 20 μm.

13. The polypeptide formulation of claim 1, wherein the polypeptide is selected from the group consisting of a growth factor, an antigen, an antibody, and an antibody drug conjugate (ADC).

14. The polypeptide formulation of claim 1, wherein the polypeptide is an antibody.

15. The polypeptide formulation of claim 1, wherein the coating increases the stability of the polypeptide.

16. The polypeptide formulation of claim 1, wherein the coating reduces the viscosity of the formulation, as compared to a formulation containing uncoated particles.

17. The polypeptide formulation of claim 1, wherein the coating slows the release rate of the polypeptide, as compared to a formulation containing uncoated particles.

18. The polypeptide formulation of claim 1, wherein the coated particle has increased hydrophilicity compared to an uncoated core.

19. The polypeptide formulation of claim 1, wherein the coated particle has increased flowability compared to an uncoated core.

20.-59. (canceled)

60. A coated particle comprising a core comprising a polypeptide enclosed by an inorganic oxide coating, the coating comprising at least one layer composed of three elements (“three element inorganic oxide layer”), wherein the three elements are: a) aluminum, zinc and oxygen; b) aluminum, silicon and oxygen; or c) silicon, zinc and oxygen.

61.-75. (canceled)

76. A method for preparing a polypeptide formulation, comprising
(1) providing coated particles comprising a core comprising a polypeptide enclosed by a coating layer comprising an organic oxide selected from the group consisting of silicon oxide, titanium oxide, zinc oxide, aluminum oxide; and
(2) mixing the coated particles with an aqueous composition, thereby creating a polypeptide formulation that comprises more than 100 mg/ml polypeptide.

77.-98. (canceled)
